(19) United States
(12) Patent Application Publication
(10) Pub. No.: US 2024/0334952 A1
(43) Pub. Date: Oct. 10, 2024

(54) USE OF LACTIC ACID BACTERIA TO IMPROVE FEED EFFICIENCY

(71) Applicants: FONTERRA CO-OPERATIVE GROUP LIMITED, Auckland (NZ); AGRESEARCH LIMITED, Hamilton (NZ)

(72) Inventors: Graeme Trevor Attwood, Hamilton (NZ); Laureen Crouzet, Hamilton (NZ); Shalome Anitta Bassett, Auckland (NZ); James William Dekker, Auckland (NZ); Jeremy Paul Hill, Auckland (NZ)

(21) Appl. No.: 18/723,369

(22) PCT Filed: Dec. 22, 2022
(86) PCT No.: PCT/IB2022/062651

§ 371 Date: Jun. 21, 2024

(30) Foreign Application Priority Data
Dec. 23, 2021 (AU) 2021904259

(51) Int. Cl. A23K 10/18; A23C 9/123; A23K 10/37; A23K 20/163; A23K 50/10; A23K 50/60; A61K 9/00; A61K 35/747; A61P 1/14

(52) U.S. Cl. CPC A23K 10/18; A23C 9/1234; A23K 10/37; A23K 20/163; A23K 50/10; A23K 50/60; A61K 9/0053; A61K 35/747; A61P 1/14

(57) ABSTRACT

This invention relates to use of a strain of probiotic bacteria or derivatives thereof for increasing feed efficiency, enhancing growth and/or productivity, improving body weight or body composition of a ruminant animal, and/or increasing milk production in a ruminant animal, inhibiting the growth of methane-producing bacteria and/or archaea in the forestomach of ruminant animals, reducing the ability of the rumen microbiome to produce methane, reducing methane emissions by a ruminant animal, and/or reducing the greenhouse gas emission footprint of a ruminant animal. Ruminant feed compositions are also provided.

### TECHNICAL FIELD

This invention relates to the use of a strain of lactic acid bacteria for increasing feed efficiency, enhancing growth and/or productivity, improving body weight or body composition of a ruminant animal, and/or increasing milk production in a ruminant animal, inhibiting the growth of methane-producing bacteria and/or archaea in the forestomach of ruminant animals, reducing the ability of the rumen microbiome to produce methane, reducing methane emissions by a ruminant animal, and/or reducing the greenhouse gas emission footprint of a ruminant animal. Ruminant feed compositions are also provided.

### BACKGROUND

Lactic acid bacteria (LAB) have been used as probiotics in humans, for a variety of benefits. LAB have also been used in animals to attempt to improve animal health and nutrition, with mixed results. They have also been investigated as an alternative to antibiotics used as growth promoters.

On the farm, LAB can be used as direct-fed microbials (DFMs), probiotics and as silage inoculants. Their actions are exerted in strain- and host-specific manners. Some studies have reported a variety of benefits, depending on the specific strains and hosts used, including a reduction in the incidence of diarrhoea, promotion of ruminal development, improved feed efficiency, increased body weight gain, and reduction in morbidity. However, their effects on performance have been mixed, and the mode of action unclear. (Krehbiel et al., 2003).

The use of LAB to reduce methane emissions from ruminants has also been proposed, with limited success.

A major source of methane emissions is the fermentation of organic matter by methanogenic bacteria and archaea. One prevalent source of anthropogenic methane emissions is in agriculture, where methane is produced by enteric fermentation in the digestive tract of ruminants, and from manure. These sources accounted for ˜30% of total global anthropogenic methane emissions in 2017 (Jackson et al., 2020). In addition, not only does methanogenesis in ruminants result in greenhouse gas emissions, but it is also energetically wasteful to the animal. It has long been recognised that methane production in ruminants dramatically impacts the efficiency with which these animals convert feed into metabolic energy. This decrease in efficiency results because methane represents a caloric loss to the ruminant of approximately 5-10% of its total caloric intake. To date however, research on the potential use of LAB to reduce methane emissions has been limited.

Thus, there remains a need for methods and compositions useful for increasing feed efficiency, enhancing growth and/or productivity, improving body weight or body composition of a ruminant animal, and/or increasing milk production in a ruminant animal. Methods and compositions for inhibiting the growth of methane-producing bacteria and/or archaea in the forestomach of ruminant animals, reducing the ability of the rumen microbiome to produce methane, reducing methane emissions by ruminant animals, and/or reducing the greenhouse gas emission footprint of ruminant animals are also desirable.

It is an object of this invention to go some way towards achieving one or more of these desiderata, or at least to offer the public a useful choice.

### SUMMARY OF THE INVENTION

In a first aspect, the invention provides isolated Lacticaseibacillus rhamnosus strain FNZ118, NMIA accession number V21/015445 dated 2 Aug. 2021, or a derivative thereof.

In one embodiment, the Lacticaseibacillus rhamnosus strain FNZ118 is a biologically pure culture.

In a second aspect, the invention provides a food or feed composition comprising Lacticaseibacillus rhamnosus strain FNZ118, NMIA accession number V21/015445 dated 2 Aug. 2021, or a derivative thereof.

In a third aspect, the invention provides a ruminant feed composition for:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal, increasing feed efficiency in a ruminant animal,
h. delivering a microorganism to a ruminant animal, and/or
i. reducing the greenhouse gas emission footprint of a ruminant animal,
   the feed composition comprising Lacticaseibacillus rhamnosus strain FNZ118, NMIA accession number V21/015445 dated 2 Aug. 2021, or a derivative thereof.

In some embodiments, the ruminant feed composition is a bovine feed composition. In some embodiments, the ruminant feed composition is a goat feed composition. In some embodiments, the ruminant feed composition is a sheep feed composition. In some embodiments, the ruminant feed composition is a bison feed composition. In some embodiments, the ruminant feed composition is a yak feed composition. In some embodiments, the ruminant feed composition is a water buffalo feed composition. In some embodiments, the ruminant feed composition is a deer feed composition. In some embodiments, the ruminant feed composition is a camel feed composition. In some embodiments, the ruminant feed composition is an alpaca feed composition. In some embodiments, the ruminant feed composition is a llama feed composition. In some embodiments, the ruminant feed composition is a wildebeest feed composition. In some embodiments, the ruminant feed composition is an antelope feed composition. In some embodiments, the ruminant feed composition is a nilgai feed composition.

In a further aspect, the invention provides a method for increasing feed efficiency in a ruminant animal, said method comprising the step of administering to said animal a food or feed composition according to the second aspect, or a ruminant feed composition according to the third aspect.

In a further aspect, the invention provides a method for enhancing the growth and/or productivity in a ruminant animal, said method comprising the step of administering to said animal a food or feed composition according to the second aspect, or a ruminant feed composition according to the third aspect.

In a further aspect, the invention provides a method for improving the body weight or body composition of a ruminant animal, said method comprising the step of administering to said animal a food or feed composition according to the second aspect, or a ruminant feed composition according to the third aspect.

In a further aspect, the invention provides a method for increasing the yield of milk and/or milk components produced from a ruminant animal, said method comprising the step of administering to said animal a food or feed composition according to the second aspect, or a ruminant feed composition according to the third aspect.

In a further aspect, the invention provides a method for inhibiting the growth of methane-producing bacteria and/or archaea in the forestomach of a ruminant animal, said method comprising the step of administering to said animal a food or feed composition according to the second aspect, or a ruminant feed composition according to the third aspect.

In a further aspect, the invention provides a method for reducing the ability of the rumen microbiome of a ruminant animal to produce methane, said method comprising the step of administering to said animal a food or feed composition according to the second aspect, or a ruminant feed composition according to the third aspect.

In a further aspect, the invention provides a method for reducing methane emissions by a ruminant animal, said method comprising the step of administering to said animal a food or feed composition according to the second aspect, or a ruminant feed composition according to the third aspect.

In a further aspect, the invention provides a method for delivering a microorganism to a ruminant animal, said method comprising the step of administering to said animal a food or feed composition according to the second aspect, or a ruminant feed composition according to the third aspect.

In a further aspect, the invention provides a method for reducing the greenhouse gas emissions of a ruminant animal, said method comprising the step of administering to said animal a food or feed composition according to the second aspect, or a ruminant feed composition according to the third aspect.

In a further aspect, the invention provides a method for increasing feed efficiency in a ruminant animal, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for enhancing the growth and/or productivity in a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for improving the body weight and/or body composition of a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for increasing the yield of milk and/or milk components produced from a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for inhibiting the growth of methane-producing bacteria and/or archaea in the forestomach of ruminant animals, wherein the method comprises administering to a ruminant animal an effective amount of Lacticaseibacillus rhamnosus strain FNZ118, NMIA accession number V21/015445 dated 2 Aug. 2021, or a derivative thereof.

In a further aspect, the invention provides a method for reducing methane emissions by a ruminant animal, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for reducing the ability of the rumen microbiome to produce methane, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for delivering a microorganism to a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for reducing the greenhouse gas emissions of a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for improving the absorptive capacity of the forestomach, for example increasing the absorptive capacity for volatile fatty acids (VFAs), wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for enhancing the physical and/or functional development of the rumen in a ruminant, for example a young ruminant, for example a young ruminant prior to weaning, wherein the method comprises administering to the animal an effective amount of Lacticaseibacillus rhamnosus strain FNZ118, NMIA accession number V21/015445 dated 2 Aug. 2021, or a derivative thereof.

In one embodiment, the method enhances anatomical development of the rumen. For example, the method enhances development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the method enhances rumen weight, ruminal wall thickness, or density of rumen papillae per cm² of ruminal wall, for example compared to an untreated animal.

In one embodiment, the method increases rumen papillae length, width, and/or surface area. For example, in some embodiments, the method increases rumen papillae length, width, and/or surface area to at least 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.22, 1.24, 1.26, 1.28, 1.30, 1.32, 1.34, 1.36, 1.38, or 1.40 times that of an untreated animal.

In one embodiment, the method enhances functional achievement of the rumen. For example, the method stimulates rumination, enhances dry matter intake (DMI), enhances absorptive ability and/or promotes maturation towards a mature physiology.

In some embodiments, the method inhibits the growth of a methylotrophic methanogen in the forestomach of the animal. In some embodiments, the method inhibits the growth of a methanogen from the genus Methanosphaera in the forestomach of the animal.

In some embodiments, the L. rhamnosus FNZ118 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus.

In a further aspect, the invention provides a composition comprising Lacticaseibacillus rhamnosus strain FNZ118, NMIA accession number V21/015445 dated 2 Aug. 2021, or a derivative thereof. In some embodiments the composition is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet, bolus, or intra-ruminal product, or the L. rhamnosus FNZ118 is encapsulated, for example in liposomes, microbubbles, microparticles or microcapsules.

In some embodiments, the L. rhamnosus FNZ118 or derivative thereof is administered in drinking water, milk, milk powder, milk replacement, milk fortifier, whey, whey powder, Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

In some embodiments, the method comprises administering to the animal the L. rhamnosus FNZ118 in an amount of at least about 10⁴ colony forming units per kilogram of dry weight carrier feed, such as at least about 10⁵, at least about 10⁶, at least about 10⁷, at least about 10⁸, at least about 10⁹, at least about 10¹⁰, at least about 10¹¹, at least about 10¹², or at least about 10¹³ colony forming units per kilogram of dry weight carrier feed. In some embodiments, the method comprises administering to the animal the L. rhamnosus FNZ118 in an amount of from 10⁴ to 10¹³ colony forming units per kilogram of dry weight carrier feed. In one embodiment, the method comprises administering to the animal the L. rhamnosus FNZ118 in an amount from 10⁸ to 10¹² colony forming units per kilogram of dry weight carrier feed.

In some embodiments, the derivative of the L. rhamnosus FNZ118 is a cell lysate of the L. rhamnosus FNZ118, a cell suspension of the L. rhamnosus FNZ118, a metabolite of the L. rhamnosus FNZ118, or a culture supernatant of the L. rhamnosus FNZ118. In some embodiments, the derivative of the L. rhamnosus FNZ118 is killed and/or non-replicating, for example heat-killed, lysed, pressure-killed, irradiated, and/or UV-treated.

In some embodiments, the method comprises further administering at least one microorganism of a different species or strain, a vaccine that inhibits methanogens or methanogenesis, and/or a natural or chemically-synthesised inhibitor of methanogenesis and/or methanogen inhibitor. An example of a useful inhibitor of methanogenesis is bromoform, which works by inhibiting the efficiency of the methyltransferase enzyme by reacting with the reduced vitamin B12 cofactor required for the penultimate step of methanogenesis.

In one embodiment, the method comprises further administering at least one microorganism of a different species or strain, a vaccine that inhibits methanogens or methanogenesis, and/or a natural or chemically-synthesised inhibitor of methanogenesis and/or methanogen inhibitor that targets a hydrogenotrophic methanogen, for example, a methanogen from the genus Methanobrevibacter.

In some embodiments, the L. rhamnosus FNZ118 or derivative thereof is administered separately, simultaneously or sequentially with one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, one or more milk-derived oligosaccharides (for example, 2′-fucosyllactose, 3′-fucosyllactose, 3′-sialyllactose, 6′-sialyllactose, lacto-N-tetraose, lacto-N-neotetraose), or any mixture of any two or more thereof.

In some embodiments, the method additionally enhances the growth or productivity of the animal, for example the method increases the yield of milk and/or milk components produced from the ruminant animal. In some embodiments, the method increases the yield of milk fat, milk protein or milk solids in the milk produced from the animal.

In some embodiments, the method additionally increases the body weight and/or improves body composition, such as altering the muscle to fat ratio, of the ruminant animal.

In some embodiments, the method additionally increases wool growth of the ruminant animal.

In some embodiments, the ruminant animal is a bovine, goat, sheep, bison, yak, water buffalo, deer, camel, alpaca, llama, wildebeest, antelope, or nilgai. In one embodiment, the ruminant animal are cattle or sheep. In one embodiment, the ruminant animal are cattle. In one embodiment, the ruminant animal is a lactating animal. In an alternative embodiment, the ruminant animal is a pre-weaning animal, such as a calf or a lamb.

In some embodiments, the ruminant feed composition is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, lick block, or molasses.

In some embodiments, the method comprises further administering at least one microorganism of a different species or strain, a vaccine that inhibits methanogens or methanogenesis, and/or a natural or chemically-synthesised inhibitor of methanogenesis and/or methanogen inhibitor.

In one embodiment, the method comprises further administering at least one microorganism of a different species or strain, a vaccine that inhibits methanogens or methanogenesis, and/or a natural or chemically-synthesised inhibitor of methanogenesis and/or methanogen inhibitor that targets a hydrogenotrophic methanogen, for example, a methanogen from the genus Methanobrevibacter.

In some embodiments, the ruminant feed composition further comprises one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

In a further aspect, the invention provides a ruminant animal to which the method of a previous aspect has been applied.

In a further aspect, the invention provides a method for producing an animal product having a reduced greenhouse gas emission footprint, the method comprising:
a. providing the ruminant animal of the previous aspect, and
b. producing an animal product from the animal.

In some embodiments, the animal product comprises dairy, meat, or wool.

In a further aspect, the invention provides a use of Lacticaseibacillus rhamnosus strain FNZ118, NMIA accession number V21/015445 dated 2 Aug. 2021, or a derivative thereof, for the manufacture of a composition for:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal,
h. delivering a microorganism to a ruminant animal, and/or
i. reducing the greenhouse gas emission footprint of a ruminant animal.

In some embodiments, the composition is or comprises a ruminant feed composition according to the third aspect.

In a further aspect, the invention provides Lacticaseibacillus rhamnosus strain FNZ118, NMIA accession number V21/015445 dated 2 Aug. 2021, or a derivative thereof, for use in:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal,
h. delivering a microorganism to a ruminant animal, and/or
i. reducing the greenhouse gas emission footprint of a ruminant animal.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE FIGURES

Embodiments of the invention will now be described with reference to the drawings in which:

FIG. 1 shows the body weight of heifers after being treated with FNZ118 (squares) or a control treatment (triangles) for the first 14 weeks of life, and subsequently moved onto pasture. The * indicates a significant difference (p<0.05) between the FNZ118 and control treatments.

### DETAILED DESCRIPTION OF THE INVENTION

The present invention is based on the finding that Lacticaseibacillus rhamnosus strain FNZ118 and derivatives thereof increase feed efficiency in ruminant animals. FNZ118 and derivatives thereof have also been shown herein to inhibit or suppress the growth of methane-producing bacteria and/or archaea in the forestomach of ruminant animals and/or reduce the ability of the rumen microbiota to produce methane. Inhibiting or suppressing the growth of methane-producing bacteria and/or archaea can reduce methane emissions and may alter the volatile fatty acid (VFA) profile, total VFA concentration, residual feed intake (RFI) and/or rate of fermentation in the rumen and forestomach, which can act as an increased energy source driving increased feed efficiency, enhanced weight gain, and/or increased productivity, such as milk, meat, or wool production, and can stimulate rumen development, such as rumen papillae development.

Accordingly, in a first aspect, the invention provides isolated Lacticaseibacillus rhamnosus strain FNZ118, NMIA accession number V21/015445 dated 2 Aug. 2021, or a derivative thereof.

In a second aspect, the invention provides a food or feed composition comprising Lacticaseibacillus rhamnosus strain FNZ118, NMIA accession number V21/015445 dated 2 Aug. 2021, or a derivative thereof.

In a third aspect, the invention provides a ruminant feed composition for:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal,
h. delivering a microorganism to a ruminant animal, and/or
i. reducing the greenhouse gas emission footprint of a ruminant animal,
   the feed composition comprising Lacticaseibacillus rhamnosus strain FNZ118, NMIA accession number V21/015445 dated 2 Aug. 2021, or a derivative thereof.

In a further aspect, the invention provides a method for increasing feed efficiency in a ruminant animal, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for enhancing the growth and/or productivity in a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for improving the body weight and/or body composition of a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for increasing the yield of milk and/or milk components produced from a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for inhibiting the growth of methane-producing bacteria and/or archaea in the forestomach of ruminant animals, wherein the method comprises administering to a ruminant animal an effective amount of Lacticaseibacillus rhamnosus strain FNZ118, NMIA accession number V21/015445 dated 2 Aug. 2021, or a derivative thereof.

In a further aspect, the invention provides a method for reducing methane emissions by a ruminant animal, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for reducing the ability of the rumen microbiome to produce methane, wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for delivering a microorganism to a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for reducing the greenhouse gas emissions of a ruminant animal, said method comprising the step of administering to said animal an effective amount of Lacticaseibacillus rhamnosus strain FNZ118, NMIA accession number V21/015445 dated 2 Aug. 2021, or a derivative thereof.

In a further aspect, the invention provides a method for improving the absorptive capacity of the forestomach, for example increasing the absorptive capacity for volatile fatty acids (VFAs), wherein the method comprises administering to the animal an effective amount of Lacticaseibacillus rhamnosus strain FNZ118, NMIA accession number V21/015445 dated 2 Aug. 2021, or a derivative thereof.

In a further aspect, the invention provides a method for enhancing the physical and/or functional development of the rumen in a ruminant, for example a young ruminant, for example a young ruminant prior to weaning, wherein the method comprises administering to the animal an effective amount of Lacticaseibacillus rhamnosus strain FNZ118, NMIA accession number V21/015445 dated 2 Aug. 2021, or a derivative thereof.

In one embodiment, the methods and compositions enhance anatomical development of the rumen. For example, the method enhances development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the methods and compositions disclosed herein enhance rumen weight, ruminal wall thickness, or density of rumen papillae per cm² of ruminal wall.

In one embodiment, the methods and compositions disclosed herein enhance functional achievement of the rumen. For example, the method stimulates rumination and/or enhances dry matter intake (DMI).

In one embodiment, the methods and compositions disclosed herein alter the abundance of heterofermentative anaerobes in the rumen microbiome. In one embodiment, the methods and compositions disclosed herein increase the abundance of heterofermentative anaerobes in the rumen microbiome.

In one embodiment the methods and compositions disclosed herein increase ruminal turnover rate and/or increase post-ruminal digestion. Without wishing to be bound by theory, it has been hypothesised that a higher rumen turnover rate selects for microorganisms that are capable of fast, heterofermentative growth on soluble sugars, producing less hydrogen, which leads to less methane formation. For example, Kamke et al (2016) note that lactate conversion to butyrate, instead of to propionate, produces 2 mol of hydrogen per hexose, which could produce 0.5 mol of methane via the hydrogenotrophic pathway, and postulate that direct fermentation of hexoses to butyrate and acetate by, for example, members of the Ruminococcaceae would produce 2.66 mol of hydrogen and allow 0.66 mol of methane to be formed. Thus, lower hydrogen production via the lactate to butyrate pathway is predicted to decrease methane production.

The term “administering” refers to the action of introducing an effective amount of L. rhamnosus strain FNZ118 or a derivative thereof into the forestomach of a ruminant animal. More particularly, this administration is an administration by oral route. This administration can in particular be carried out by supplementing the feed or drink intended for the animal with the strain; the supplemented feed or drink then being ingested by the animal.

The term “effective amount” refers to a quantity of L. rhamnosus strain FNZ118 or a derivative thereof sufficient to allow a desired effect, i.e., inhibition of the growth of methane-producing bacteria and/or archaea in the forestomach of the animal, a reduction in methane emissions by the animal, or an increase in feed efficiency in the animal, in comparison with a reference. The desired effect (such as inhibition of growth of methane-producing bacteria and/or archaea and/or reduction of methane production or emission) can be measured in vitro or in vivo. For example, the desired effect can be measured in vitro using the methods described herein, for example, in the Examples below, in an artificial rumen system, such as that described in T. Hano (1993) J. Gen. Appl. Microbiol., 39, 35-45, or by in vivo oral administration to ruminants.

This effective amount can be administered to the ruminant animal in one or more doses.

The terms “reducing methane production” and “reducing methane emissions”, e.g., “reducing methane production by the animal” and “reducing methane emissions by the animal” refers to reducing methane production or emissions by any mechanism, and from any ruminant-related source. For example, the term may refer to a reduction in methane produced within the forestomach of ruminant animals, or it may refer to a reduction in methane produced or emitted by the faeces of a ruminant animal.

It is anticipated that the reduction in methane production may be due to a variety of mechanisms. These may include, for example, killing methanogens (i.e. a bactericidal/archaeacidal effect), inhibiting the growth of methanogens (i.e. a bacteriostatic/archaeostatic effect), and/or inhibiting the ability of the forestomach or rumen microbiota to produce methane. Inhibiting the ability of the forestomach or rumen microbiota to produce methane may be via a variety of mechanisms, including, for example, physical and/or chemical changes to the forestomach or rumen environment, changes to the microbiota, the inhibition of one or more methanogenic pathways, and/or cross-feeding (or disrupting cross-feeding) of intermediaries between members of the microbiome.

It will be appreciated that a reduction in greenhouse gas (GHG) emissions, such as methane emissions, is desirable. GHG emissions may be reduced either directly, for example by reducing the ability of the rumen microbiome to produce methane and/or by reducing methane emissions by a ruminant animal, or indirectly. One example of indirect reduction in GHG emissions is by altered land use and/or land retirement. Animals with improved feed efficiency (such as animals to which the methods or compositions of the present invention have been applied) may require less pasture for forage and/or less imported feed. Alternatively or additionally, more animals may be able to be farmed on a given land area, allowing the same production with reduced land use. In either case, decreased land requirements may allow unused pasture to be retired, for example by planting trees or other vegetation for carbon sequestration. Such land use changes may further lower the GHG emissions per farm, resulting in a lower GHG emission footprint per animal, and/or per kg animal product (such as milk, meat, or wool).

The GHG emission footprint of an animal and/or animal product may be determined using techniques known in the art. It will be appreciated that certain GHGs produce more global warming potential than others. For example, 1 kg of methane emissions produces a global warming impact approximately equivalent to 25 kg of CO₂. To account for this, GHG emissions are typically reported as CO₂ equivalents (CO₂ₑ), i.e. the amount of CO₂ that would have an equivalent global warming impact. The GHG emission footprint may be calculated per animal, or per amount of animal product (for example, per kg milk solids, per kg meat, or per kg wool). As described above, the GHG emission footprint should take into account alterations to land use, such as planting trees or other vegetation for carbon sequestration.

The term “animal product” refers to any product produced from or by an animal, or containing any animal-derived component(s). The term is intended to include products that are directly produced by an animal (for example, milk, meat, and wool) and products that include or are made from animal ingredients, that have optionally undergone further processing, optionally with other ingredients. For example, the term is intended to include foods and beverages that contain animal ingredients, such as various dairy products (including buttermilk, cheese, cream, formula, ice cream, milk, milk powder, puddings, shakes, smoothies, and yoghurts), meat products (such as a chops, ground meat, hamburger, sausages, sausage meat, and steaks) and other products that contain animal ingredients.

The term “feed efficiency” refers to the relationship between feed intake and muscle weight gain or milk yield. Microbial fermentation in the forestomach or rumen produce volatile fatty acids (VFA) such as acetic acid, propionic acid and butyric acid. These fatty acids are absorbed directly from the rumen wall and used as raw materials for growth and development of the animal, milk components, and other final digested products. The majority of the energy consumed by body tissues is used to produce milk or milk components, or muscle. Thus, when the utilisation of energy is improved, milk production, e.g., milk yield, and/or milkfat, milk protein, and/or milk solids can be increased. Increases in muscle, and/or improvements in body composition, such as altered muscle/fat ratio in an animal, can also be achieved.

Feed efficiency can be calculated by dividing the weight of milk produced by an animal, or the liveweight of an animal, by the weight of dry matter consumed by that animal. Thus, an animal with a higher feed efficiency will produce more milk, milk with a higher content of milk components such as, but not limited to, fat and protein, and/or will show increased weight gain compared to an animal with a lower feed efficiency when given the same nutrient input. Feed efficiency can be measured by differences in the growth of an animal by any of the following parameters: average daily weight gain, total weight gain, feed conversion, which includes both feed:gain and gain:feed, feed efficiency, mortality, and feed intake. That is to say, improved feed efficiency can mean that the ratio of feed intake/muscle weight gain is decreased. Improved feed efficiency can also mean that the ratio of muscle weight gain/feed intake is increased. The term feed efficiency may also refer to the feed intake/weight gain or weight gain/feed intake. The feed efficiency may be standardised to account for differences in protein and fat content by using the energy-corrected milk (ECM) yield instead of the weight of milk. This can be calculated using the following formula (Tyrrell and Reid, 1965):

$$ECM = ( 12.82 \times weight of fat in pounds ) + ( 7.13 \times weight of protein in pounds ) + ( 0.323 \times weight of milk in pounds ) .$$

“Feed conversion” and “residual feed intake (RFI)” are also commonly used measures of feed efficiency, and the terms are often used almost interchangeably. In animal husbandry, feed conversion ratio or feed conversion rate is a ratio or rate measuring of the efficiency with which the bodies of livestock convert animal feed into the desired output. RFI is defined as the difference between the actual dry matter intake (DMI) of an animal and the expected DMI required for maintenance and growth.

The primary advantage of improving feed efficiency (i.e., improving the feed conversion ratio or lowering RFI) is to reduce DMI in animals without compromising growth performance, because feed-related costs often represent the largest production expense in beef or milk production. Any reduction in DMI to produce a unit of beef or milk product would minimise feed costs, resulting in maximising the overall profitability of the beef or dairy industry.

In one embodiment, the feed efficiency in a ruminant animal is increased to at least about 1.01× of the feed efficiency of an untreated animal, such as at least about 1.02×, 1.03×, 1.04×, 1.05×, 1.06×, 1.07×, 1.08×, 1.09×, 1.10×, 1.12×, 1.14×, 1.16×, 1.18×, such as at least about 1.20×.

Increased feed efficiency may result from alteration of the volatile fatty acid (VFA) profile, total VFA concentration and/or the rate of fermentation in the rumen and forestomach.

In some embodiments, L. rhamnosus strain FNZ118 or a derivative thereof promotes propionic acid production. Propionic acid has higher ATP production efficiency compared with other volatile fatty acids, and hence, feed efficiency is improved owing to the promotion of propionic acid production. Propionic acid is also glucogenic and can thus promote lactose synthesis in the mammary gland.

In some embodiments, L. rhamnosus FNZ118 or a derivative thereof shifts hydrogen metabolism from methanogenesis to short chain/volatile fatty acid (VFA) production, for example to propionic acid production. Propionate is predominantly used as a glucose precursor in ruminants, and more propionate formation would likely result in a more efficient utilisation of feed energy. Maximizing the flow of metabolic hydrogen in the forestomach or rumen away from methane and toward VFA (mainly propionate) would increase the efficiency of ruminant production and decrease its environmental impact, and would enhance rumen development and/or rumen papillae development.

Acetate is the primary substrate for mammary lipid synthesis, along with β-hydroxybutyrate which is produced during the absorption of butyrate. Consequently, a high acetate fermentation pattern will provide substrate to maintain or increase milk fat.

Thus, in some embodiments, L. rhamnosus strain FNZ118 or a derivative thereof results in an increase in milkfat, milk protein, overall milk volume and/or milk solids as a result of increased VFAs in the forestomach or rumen, which can act as an increased energy source driving increased production.

In some embodiments, the yield of milk and/or or milk components produced from the animal are preferably increased by 1.5% or more, more preferably, by 3.0% or more, by 4.5% or more, or by 6.0% or more.

In some embodiments, L. rhamnosus strain FNZ118 or a derivative thereof results in an increase in liveweight, muscle mass, and/or fat deposition, and/or improvements in body composition, such as altered muscle/fat ratio in an animal as a result of increased VFAs in the forestomach or rumen, which can act as an increased energy source driving increased production.

In some embodiments, the liveweight of the animal is preferably increased by 1% or more, more preferably, by 2% or more, by 3% or more, by 4% or more, by 5% or more, by 6% or more, by 7% or more, by 8% or more, by 9% or more, or by 10% or more, in comparison to a reference animal.

It is anticipated that the present invention could also be used to extend the lactation cycle of a lactating ruminant, such as a cow. A cow directs a significant portion of its energy towards producing milk during lactation. After a long period of lactation, its body condition will be poorer for it. Because of this, the lactation period is usually shortened or curtailed to prevent excess deterioration on body condition. It is anticipated that the methods and ruminant feed composition disclosed herein will increase feed efficiency by the ruminant animal and therefor reduce the impact of milk production on body condition. As a result, it would be possible to milk cows for a longer duration.

It is also anticipated that the present invention could also be used to reduce or ameliorate the deterioration of body condition due to lactation. It is anticipated that the methods and ruminant feed compositions disclosed herein will increase feed efficiency by the ruminant animal and therefore result in the ruminant animal having an improved body condition at the end of lactation. For example, the animal has a higher body condition score (BCS) when the animal enters the dry period. As a result, the ruminant animal would require less dry matter intake during the offseason to gain body condition. Alternatively or additionally, the methods and ruminant feed compositions disclosed herein are useful for improving body condition of an animal prior to lactation. For example, the methods and compositions disclosed herein could improve the body composition of the mother and/or the foetus or neonate. For example, the methods and compositions disclosed herein could improve body composition and/or weight of the neonate at birth.

It is also anticipated that the present invention could be similarly useful for reducing or ameliorating the deterioration of body condition in other times of stress, such as calving, drought, or insufficient feed intake.

Liveweight and Body Condition Scores are commonly used in the industry as measures of animal growth and performance.

Live weight is an objective measure used to assess animal growth, and is one of the best measures of animal performance. It is the primary measure the dairy industry uses to indicate how well-grown dairy heifers are. Significant research has focused on the relationship between liveweight and performance, particularly heifer (cow) performance, and led to the identification of liveweight targets. Achieving liveweight targets will optimise a heifer's lifetime performance, increase stock longevity and the return on investment in the farming business.

In New Zealand, the liveweight targets for heifers are 30, 60 and 90% of mature liveweight at 6, 15 (pre-mating) and 22 (pre-calving) months old. The 22 month target includes an adjustment for pregnancy. Heifers grown to liveweight targets are also more likely to meet body condition score (BCS) targets at calving, which contributes to better milk production in the first lactation.

Body condition score (BCS) is a subjective measure used for assessing animal performance and ensuring animal welfare is maintained. The industry standard dairy cow BCS scale applies to heifers from 20 months of age onwards. The body condition score (BCS) target for heifers at 22 months of age (pre-calving) is 5.5.

There is a well-established relationship between liveweight and milk production. Accordingly, one of the primary benefits of achieving liveweight targets is increasing milk yield.

Heifer liveweight prior to calving has been demonstrated to have a significant effect on milk production in various studies, both in New Zealand (Handcock et al. 2019; McNaughton, LR and T Lopdell. 2013; MacDonald et al. 2005; van der Waaij et al. 1997) and overseas (Carson et al. 2002; Dobos et al. 2001).

If a 9% milk solids test is assumed, the expected response is about two kilograms of milk solids per lactation for every one percent increase in target liveweight attained. For a heifer with a pre-calving liveweight target of 500 kg, five kilograms is equal to one percent of liveweight. Van der Waaij et al. (1997) reported a response of 6 L of milk and 0.43 kg of milksolids per kg of liveweight, whilst Dobos et al. (2001) reported 5.35 litres of milk and 0.42 kg of milksolids per kg of liveweight at first calving. Multiplying these values by 5, for a 5 kg advantage, gives 26.8-30 L of milk and 2.1-2.15 kg of milk solids for every 1% increase in live weight attained. A similar response was reported by McNaughton, LR and T Lopdell (2013), in which in pre-calving heifers, every 1% increase in the percentage of target liveweight attained was associated (P<0.001) with an increase in milk volume of 23±0.6 litres in the first lactation and 24±0.9 litres in the second lactation.

Thus, in some embodiments, the methods and compositions disclosed herein increase milk production, for example the yield of milk and/or milk components produced from the ruminant animal. In some embodiments, the methods and compositions disclosed herein increase the yield of milk fat, milk protein or milk solids in the milk produced from the animal.

In some embodiments, the methods and compositions disclosed herein increase first-lactation milk production. In some embodiments, the methods and compositions disclosed herein increase accumulated milk production over multiple lactations, for example over the first two, or first three lactations. In some embodiments, the methods and compositions disclosed herein increase accumulated milk production over all lactation periods of the animal.

In some embodiments, milk production of the animal is preferably increased by 1% or more, more preferably, by 2% or more, by 3% or more, by 4% or more, or by 5% or more, in comparison to an untreated or reference animal.

In some embodiments, the milk production of the animal is preferably increased by 5 kg or more of milk solids per lactation, more preferably by 6 kg or more, by 7 kg or more, by 8 kg or more, by 9 kg or more, by 10 kg or more, by 11 kg or more, by 12 kg or more, or by 13 kg or more of milk solids per lactation, in comparison to an untreated or reference animal.

In some embodiments, the milk production of the animal is preferably increased by 60 L or more of milk solids per lactation, more preferably by 70 L or more, by 80 L or more, by 90 L or more, by 100 L or more, by 110 L or more, by 120 L or more, or by 130 L or more per lactation, in comparison to an untreated or reference animal.

As discussed above, the methods and compositions disclosed herein enhance the physical and/or functional development of the rumen, particularly in early life of young or pre-weaning ruminants. The development of the rumen involves three distinct processes: (i) anatomical development (e.g., growth in rumen mass and growth of rumen papillae), (ii) functional achievement (e.g., fermentation capacity and enzyme activity), and (iii) microbial colonization (bacteria, fungi, methanogens, and protozoa).

The anatomical development of the rumen is a process that occurs following three phases: non-rumination (0-3 weeks), transitional phase (3-8 weeks), and rumination (from 8 weeks on. During the transitional phase, growth and development of the ruminal absorptive surface area (papillae) is essential to enable absorption and utilisation of digestion end products, specifically rumen volatile fatty acids. The presence and absorption of volatile fatty acids stimulates rumen epithelial metabolism and may be key in initiating rumen epithelial development. A continuous exposure to volatile fatty acids maintains rumen papillae growth, size, and function. Different volatile fatty acids stimulate such development differently, with butyrate the most stimulatory, followed by propionate. Thus, it is expected that shifts hydrogen metabolism from methanogenesis to short chain/volatile fatty acid (VFA) production, for example to propionic acid production, would therefore enhance rumen epithelial growth and development.

### Ruminants

Ruminants are a group of herbivores having a stomach comprising multiple compartments, that digest their food by a first microbial fermentation in the rumen to form a cud, regurgitating and chewing the cud, and then swallowing the chewed cud for further digestion. This group includes, but is not limited to, the Ruminantia and Tylopoda suborders, and includes several species of domesticated livestock. In one embodiment, the ruminant animal is a bovine, goat, sheep, bison, yak, water buffalo, deer, camel, alpaca, llama, wildebeest, antelope, or nilgai. In a preferred embodiment, the ruminant animal is a bovine or a sheep.

In one embodiment, the ruminant animal is a lactating animal. In an alternative embodiment, the ruminant animal is a pre-weaning animal, such as a calf or a lamb.

The ruminant stomach is divided into the nonglandular forestomach (rumen, reticulum, omasum) and the terminal glandular stomach, the abomasum.

In some embodiments, the ruminant animal is neonatal, newborn, or young. For example, in some embodiments, the ruminant animal is one day, 2 days, 3 days, 4 days, 5 days, 6 days, 1 week, 2 weeks, 3 weeks, one month, or 2 months of age.

In some embodiments, L. rhamnosus strain FNZ118 or a derivative thereof is administered to the ruminant animal prior to weaning. In some embodiments, the L. rhamnosus FNZ118 or derivative thereof is administered to the ruminant animal after weaning. In some embodiments, the L. rhamnosus FNZ118 or derivative thereof is administered to the ruminant animal both prior to weaning and after weaning. For example, in some embodiments, L. rhamnosus strain FNZ118 or a derivative thereof is administered throughout the ruminant animal's life.

For example, the L. rhamnosus FNZ118 or derivative thereof is administered to the ruminant animal on or about day 0 of birth, for example around day 0, day 1 or day 2 of birth. Administration may then occur at least one per day, for example multiple times per day, sufficient to obtain persistency of effect. For example, administration may continue for 2 days, 3 days, 4 days, 5 days, 6 days, 1 week, 2 weeks, 3 weeks, one month, 6 weeks, 2 months, 10 weeks or three months or more from birth. In some embodiments, the administration of L. rhamnosus strain FNZ118 or a derivative thereof continues for four months, five months, six months, seven months, eight months, nine months, ten months, eleven months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or for the life of the ruminant animal.

Lacticaseibacillus rhamnosus FNZ118

A culture of Lacticaseibacillus rhamnosus FNZ118 (also known as Lactobacillus rhamnosus FNZ118) was isolated from a human source and deposited at the National Measurement Institute of Australia (NMIA), 1/153 Bertie Street, Port Melbourne, Victoria, Australia 3207 on 2 Aug. 2021, and was given accession number V21/015445. This is a recognised International Depositary Authority under the Budapest Treaty on the International Recognition of the Deposit of Microorganisms for the Purposes of Patent Procedure. The terms Lactobacillus rhamnosus strain FNZ118, Lactobacillus rhamnosus FNZ118, Lacticaseibacillus rhamnosus FNZ118, L. rhamnosus FNZ118, and FNZ118 are used interchangeably herein.

Whole genome sequencing using a combination of short-read (Illumina) and long-read (MinION/ONT) sequencing technologies was used to create hybrid genome assemblies. The final hybrid assembly contained 7 contigs. Total length was 2997051 bp (3.0 Mb). Species ID was confirmed for the FNZ118 strain as Lacticaseibacillus rhamnosus using the taxonomic sequence classifier programme, Kraken.

All WGS and associated bioinformatics were conducted in accordance with the EFSA Guidance available at:
https://efsa.onlinelibrary.wiley.com/doi/pdf/10.2903/j.efsa.2018.5206 in conjunction with the latest EFSA statement (July 2021) available at:
https://efsa.onlinelibrary.wiley.com/doi/full/10.2903/j.efsa.2021.6506.

### Morphological Properties

The morphological properties of L. rhamnosus FNZ118 are described below.

Short to medium rods with square ends in chains, generally 0.7×1.1×2.0-4.0 μm, when grown in MRS broth. Gram positive, non-mobile, non-spore forming, catalase negative facultative anaerobic rods.

### Further Characterisation

It will be appreciated that there are a wide variety of methods known and available to the skilled artisan that can be used to confirm the identity of L. rhamnosus FNZ118, wherein exemplary methods include DNA fingerprinting, genomic analysis, sequencing, and related genomic and proteomic techniques.

L. rhamnosus Strain FNZ118 and Derivatives Thereof

As described herein, certain embodiments of the present invention utilise live L. rhamnosus strain FNZ118. In other embodiments, a derivative of L. rhamnosus strain FNZ118 is utilised.

As used herein, the term “derivative” and grammatical equivalents thereof when used with reference to bacteria (including use with reference to a specific strain of bacteria such as L. rhamnosus FNZ118) contemplates mutants and homologues of or derived from the bacteria, killed or attenuated bacteria such as but not limited to heat-killed, lysed, fractionated, pressure-killed, irradiated, and UV- or light-treated bacteria, and material derived from the bacteria including but not limited to bacterial cell wall compositions, bacterial cell lysates, lyophilised bacteria, anti-methanogen factors from the bacteria, bacterial metabolites, bacterial cell suspensions, bacterial culture supernatant, and the like, wherein the derivative retains anti-methanogen activity. Transgenic microorganisms engineered to express one or more anti-methanogen factors are also contemplated. Methods to produce such derivatives, such as but not limited to one or more mutants of L. rhamnosus strain FNZ118 or one or more anti-methanogen factors, and particularly derivatives suitable for administration to a ruminant animal (for example, in a composition) are well known in the art.

It will be appreciated that methods suitable for identifying L. rhamnosus strain FNZ118, such as those described above, are similarly suitable for identifying derivatives of L. rhamnosus strain FNZ118, including for example mutants or homologues of L. rhamnosus strain FNZ118, or for example bacterial metabolites from L. rhamnosus strain FNZ118.

The term “anti-methanogen factor” refers to a bacterial molecule responsible for mediating anti-methanogen activity, including but not limited to bacterial DNA motifs, RNA including mRNA and miRNA, proteins, exosomes, bacteriocins, bacteriocin-like molecules, anti-microbial peptides, antibiotics, antimicrobials, small molecules, polysaccharides, or cell wall components such as lipoteichoic acids and peptidoglycan, or a mixture of any two or more thereof. While, as noted above, these molecules have not been clearly identified, and without wishing to be bound by any theory, their presence can be inferred by the presence of anti-methanogen activity.

The term “anti-methanogen activity” refers to the ability of certain microorganisms to inhibit the growth of methanogenic bacteria and/or archaea, and/or to reduce the production of methane by methanogenic bacteria and/or archaea. This ability may be limited to inhibiting the growth of and/or ability to produce methane of certain groups of methanogenic bacteria and/or archaea such as, for example, inhibiting the growth of hydrogenotrophic methanogens, inhibiting the ability of hydrogenotrophic methanogens to produce methane, inhibiting the growth of methylotrophic methanogens, inhibiting the ability of methylotrophic methanogens to produce methane, inhibiting the growth of certain species of methanogens, or inhibiting the ability of certain species of methanogens to produce methane.

Reference to retaining anti-methanogen activity is intended to mean that a derivative of a microorganism, such as a mutant or homologue of a microorganism or an attenuated or killed microorganism, or a cell culture supernatant, still has useful anti-methanogen activity, or that a composition comprising a microorganism or a derivative thereof still has useful anti-methanogen activity. While the bacterial molecules responsible for mediating anti-methanogen activity have not been clearly identified, molecules that have been proposed as possible candidates include bacterial DNA motifs, RNA including mRNA and miRNA, proteins, exosomes, bacteriocins, antibiotics, surface proteins, small organic acids, polysaccharides, and cell wall components such as lipoteichoic acids and peptidoglycan. It has been postulated that these interact with components of the methanogenic bacteria and/or archaea to give a growth-inhibitory effect. Preferably, the retained activity is at least about 35, 40, 45, 50, 55, 60, 65, 70, 75, 80, 85, 90, 95, 99 or 100% of the activity of an untreated (i.e., live or non-attenuated) control, and useful ranges may be selected between any of these values (for example, from about 35 to about 100%, from about 50 to about 100%, from about 60 to about 100%, from about 70 to about 100%, from about 80 to about 100%, and from about 90 to about 100%).

Using conventional solid substrate and liquid fermentation technologies well known in the art, L. rhamnosus strain FNZ118 can be grown in sufficient amounts to allow use as contemplated herein. For example, L. rhamnosus strain FNZ118 can be produced in bulk for formulation using nutrient film or submerged culture growing techniques, for example under conditions as described in WO99/10476. Briefly, growth is carried out under aerobic conditions at any temperature satisfactory for growth of the organism. For example, for L. rhamnosus strain FNZ118, a temperature range of from 30 to 40° C., preferably 37° C., is preferred. The pH of the growth medium is slightly acidic, preferably about 6.0 to 6.5. Incubation time is sufficient for the isolate to reach a stationary growth phase.

Bacterial cells may be harvested by methods well known in the art, for example, by conventional filtering or sedimentary methodologies (e.g. centrifugation) or harvested dry using a cyclone system. Bacterial cells can be used immediately or stored, preferably freeze-dried or chilled at −20° to 6° C., preferably −4° C., for as long as required using standard techniques. Cryoprotectants, cryopreservatives, and/or lyoprotectants may also be used to enhance the stability and/or viability of bacterial cells when dried and/or frozen as is known in the art.

### Supernatants

Further embodiments of the present invention utilise supernatant(s) from a cell culture comprising L. rhamnosus strain FNZ118 or a derivative thereof. These embodiments include processes for preparing a bacterial culture supernatant, said process comprising culturing bacterial cells, and separating the supernatant from the cultured cells, thereby obtaining the supernatant. This process also enables further isolation of bacterial molecules responsible for mediating anti-methanogen activity that are obtainable from the supernatant.

As would be understood by the skilled addressee, a supernatant useful in the present invention encompasses both the supernatant from such cultures, and/or concentrates of such supernatant and/or fractions of such supernatant.

The term “supernatant” in the present context refers to a medium from a bacterial culture from which the bacteria have subsequently been removed, e.g. by centrifugation or filtration.

A supernatant useful in the present invention can readily be obtained by a simple process for preparing a bacterial culture supernatant, said process comprising
a) culturing cells of L. rhamnosus strain FNZ118,
b) optionally releasing of active compounds and/or extracellular components of the cells by various cellular treatments such as, but not limited to, acidic or alkaline modifications, sonication, detergents e.g. Sodium dodecyl sulfate (SDS) and/or Triton X, muralytic enzymes e.g. mutalolysin and/or lysozyme, salt and/or alcohol, and
c) separating the supernatant from the cultured cells,
   thereby obtaining said supernatant.

In a preferred embodiment of this process, the supernatant composition is further subjected to a drying step to obtain a dried culture product.

The drying step may conveniently be freeze drying or spray drying, but any drying process which is suitable for drying of anti-methanogen factors such as bacteriocins, also including vacuum drying and air drying, are contemplated.

Although the content of the supernatants produced by L. rhamnosus strain FNZ118 is not yet characterised in detail, it is known that certain bacterial strains may produce bacteriocins that are small heat-stable proteins and therefore, without wishing to be bound by theory, it is expected that even drying methods, including spray drying, which result in moderate heating of the culture eluate product, will result in active compositions, as demonstrated in the Examples described herein.

### Lysate

A fluid containing the contents of lysed cells is called a lysate. A lysate contains active components of the bacterial cells and may be either crude, thus containing all cellular components, or partially and/or completely separated in separate fractions, such as extracellular components, intracellular components, proteins etc.

Methods for producing bacterial cell lysates are well known in the art. Such methods can include, but are not limited to, mechanical lysis, such as mechanical shearing, grinding, milling, or sonication, enzymatic lysis, such as by enzymes that degrade the bacterial cell wall, chemical lysis, such as using detergents, denaturants, pressure alterations, and/or osmotic shock, and combinations of the above.

Further embodiments of the present invention thus utilise a lysate of L. rhamnosus strain FNZ118 or a derivative thereof.

### Cell Suspension

The present invention may also in some embodiments utilise a cell suspension comprising L. rhamnosus strain FNZ118 or a derivative thereof.

In the present context, the term “cell suspension” relates to a number of cells of L. rhamnosus strain FNZ118 or a derivative thereof dispersed or in suspension in a liquid e.g. a liquid nutrient medium, culture medium or saline solution.

The cells may be presented in the form of a cell suspension in a solution that is suitable for dispersion. The cell suspension can e.g. be dispersed via spraying, dipping, or any other application process.

The cells may be viable, but the suspension may also comprise inactivated or killed cells or a lysate hereof. In one embodiment, the suspension of the present invention comprises viable cells. In another embodiment, the suspension of the present invention comprises inactivated, killed or lysed cells.

### Bacteriocins

Bacteriocins are antimicrobial compounds produced by bacteria to inhibit other bacterial strains and species.

Lactic acid bacteria (LAB) are well known to produce bacteriocins and these compounds are of global interest to the food industry because they inhibit the growth of many spoilage and pathogenic bacteria, thus extending shelf life and safety of foods. Bacteriocins are typically considered to be narrow spectrum antibiotics. Moreover, bacteriocins of especially LAB display very low human toxicity and have been consumed in fermented food for millennia.

A further aspect of the invention provides an isolated antimicrobial compound obtainable from L. rhamnosus strain FNZ118 or a derivative thereof. Such antimicrobial compound may for example be obtained from a supernatant or lysate resulting from the process described herein, further comprising an isolation step.

As is illustrated in the Examples disclosed herein, it has been found that L. rhamnosus strain FNZ118 and/or compositions comprising L. rhamnosus strain FNZ118, and/or the culture supernatant of L. rhamnosus strain FNZ118 are useful as an antimicrobial compound, in particular for inhibiting the growth of methane-producing bacteria and/or inhibiting the ability of methanogens to produce methane.

In the present context, the term antimicrobial compound utilises a compound that kills microorganisms, impair their survival or inhibits their growth.

Antimicrobial compounds can be grouped according to the microorganisms they act primarily against. For example, antibacterials are used against bacteria and antifungals are used against fungi. They can also be classified according to their function. Compounds that kill microbes are called microbicidal, while those that merely inhibit their growth are called microbiostatic.

In one embodiment, the present invention relates to an antimicrobial compound, which is microbicidal. In another embodiment, the present invention relates to an antimicrobial compound, which is microbiostatic. In another embodiment, the present invention relates to an antimicrobial compound, which is antibacterial.

### Ruminant Feed or Carrier Compositions

A ruminant feed composition useful herein may be formulated as a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus. Appropriate formulations may be prepared by an art skilled worker with regard to that skill and the teaching of this specification.

The composition can be administered as a top dressing on, or mixed into, a standard feed material such as a daily ration. In addition, the strain can be administered in a partial or total mixed ration (TMR), pelleted feedstuff, mixed in with liquid feed or drink, mixed in a protein premix, or delivered via a vitamin and mineral premix.

In one embodiment, compositions useful herein include any edible feed product which is able to carry bacteria or a bacterial derivative. As used in this application, the term “feed(s)” or “animal feed(s)” refers to material(s) that are consumed by animals and contribute energy and/or nutrients to an animal's diet. Animal feeds typically include a number of different components that may be present in forms such as concentrate(s), premix(es), co-product(s), or pellets. Examples of feeds and feed components include Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, and molasses. Other compositions useful as a carrier include milk, milk powder, milk replacement, milk fortifier, colostrum, whey, whey powder, sucrose, maltodextrin, rice hulls and the like.

In certain embodiments, the feed composition is formed through a process of growing L. rhamnosus strain FNZ118 using a milk-based carrier, such as thermalized milk, or a non-milk-based carrier, to create a fermented yoghurt-style composition. Methods to create such fermented yoghurt-style compositions are well known in the art, and may include, for example, using a warm water bath or other heating means to incubate the milk at a suitable temperature until a sufficient cell density is reached, such as over 12 hours. In one embodiment, the temperature is 25-30° C. Optionally, the milk may include other additives to promote bacterial growth, such as yeast extract. In certain embodiments, this method takes place on-site, such as on the farm where the probiotic feed supplementation is to take place. The fermented yoghurt-style composition may be administered by oral application, such as by drenching. In some embodiments, the fermented yoghurt-style composition is administered at a dose of 1-100 ml per day, such as 2-50, 5-30, or 10-20 ml per day.

Other suitable feed formulations for ruminants are described in E. W. Crampton et al., Applied Animal Nutrition, W. H. Freeman and Company, San Francisco, CA., 1969 and D. C. Church, Livestock Feeds and Feeding, 0 & B Books, Corvallis, Oreg., 1977, both of which are incorporated herein by reference.

In one embodiment, compositions useful herein include any non-feed carrier consumed by the animal to which bacteria or a bacterial derivative is added, such as vermiculite, zeolites or crushed limestone and the like.

In certain embodiments, the composition of the invention comprises live L. rhamnosus strain FNZ118. Methods to produce such compositions are well known in the art.

In some embodiments, the composition of the invention comprises one or more derivatives of L. rhamnosus strain FNZ118. Again, methods to produce such compositions are well known in the art and may utilise standard microbiological and pharmaceutical practices. In some embodiments, the composition comprises a dried culture product, such as a supernatant or cell lysate as described herein.

It will be appreciated that a broad range of additives or carriers may be included in such compositions, for example to improve or preserve bacterial viability or to increase anti-methanogen activity of L. rhamnosus strain FNZ118 or a derivative thereof. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

In some embodiments, the L. rhamnosus FNZ118 or derivative thereof are encapsulated. Methods to produce such encapsulated bacteria are well known in the art. In some embodiments, the L. rhamnosus FNZ118 or derivative thereof are encapsulated in liposomes, microbubbles, microparticles or microcapsules and the like. Such encapsulants can include natural, semisynthetic, or synthetic polymers, waxes, lipids, fats, fatty alcohols, fatty acids, and/or plasticisers, for example alginates, gums, K-Carrageenan, chitosan, starch, sugars, gelatine, and so on.

In certain embodiments, the L. rhamnosus strain FNZ118 is in a reproductively viable form and amount.

The composition may comprise a carbohydrate source, such as a disaccharide including, for example, sucrose, fructose, glucose, or dextrose. Preferably the carbohydrate source is one able to be aerobically or anaerobically utilised by L. rhamnosus strain FNZ118.

In such embodiments, the composition preferably is capable of supporting reproductive viability of the L. rhamnosus strain FNZ118 for a period greater than about two weeks, preferably greater than about one month, about two months, about three months, about four months, about five months, more preferably greater than about six months, most preferably at least about 2 years to about 3 years or more.

In certain embodiments, an oral composition is formulated to allow the administration of an effective amount of L. rhamnosus strain FNZ118 to establish a population in the gastrointestinal tract of the animal when ingested. The established population may be a transient or permanent population.

While various routes and methods of administration are contemplated, oral administration of L. rhamnosus strain FNZ118, such as in a composition suitable for oral administration, is currently preferred. It will of course be appreciated that other routes and methods of administration may be utilised or preferred in certain circumstances.

The term “oral administration” includes oral, buccal, enteral, intra-ruminal, and intra-gastric administration.

In theory one colony forming unit (cfu) should be sufficient to establish a population of L. rhamnosus strain FNZ118 in an animal, but in actual situations a minimum number of units are required to do so. Therefore, for therapeutic mechanisms that are reliant on a viable, living population of probiotic bacteria, the number of units administered to a subject will affect efficacy.

In one embodiment, a composition formulated for administration will be sufficient to provide at least about 6×10⁹ cfu L. rhamnosus strain FNZ118 per day, for example at least about 6×10¹¹ cfu per day. In another embodiment, a composition formulated for administration will be sufficient to provide at least about 10¹² cfu L. rhamnosus strain FNZ118 per day.

Methods to determine the presence of a population of gut and/or rumen flora, such as L. rhamnosus strain FNZ118, in the gastrointestinal tract of a subject are well known in the art, and examples of such methods are presented herein. In certain embodiments, presence of a population of L. rhamnosus strain FNZ118 can be determined directly, for example by analysing one or more samples obtained from an animal and determining the presence or amount of L. rhamnosus strain FNZ118 in said sample. In other embodiments, presence of a population of L. rhamnosus strain FNZ118 can be determined indirectly, for example by observing a reduction in methane emissions or methane production, a reduction in hydrogen production, or a decrease in the number of other gut and/or rumen flora in a sample obtained from an animal. Combinations of such methods are also envisaged.

The efficacy of a composition useful according to the invention can be evaluated both in vitro and in vivo. See, for example, the examples below. Briefly, the composition can be tested for its ability to inhibit the growth of methanogenic bacteria and/or archaea, or its ability to reduce the production of methane by methanogenic bacteria and/or archaea. For in vivo studies, the composition can be fed to or injected into a ruminant and its effects on ruminal methanogenic bacteria and/or archaea, and its effect on methane emissions are then assessed. Based on the results, an appropriate dosage range and administration route can be determined.

Methods of calculating appropriate dose may depend on the nature of the active agent in the composition. For example, when the composition comprises live bacteria, the dose may be calculated with reference to the number of live bacteria present. For example, as described herein the examples the dose may be established by reference to the number of colony forming units (cfu) to be administered per day, or by reference to the number of cfu per kilogram dry feed weight.

By way of general example, the administration of from about 1×10⁶ cfu to about 1×10¹² cfu of L. rhamnosus strain FNZ118 per kg dry feed weight per day, preferably about 1×10⁶ cfu to about 1×10¹¹ cfu/kg/day, about 1×10⁶ cfu to about 1×10¹⁰ cfu/kg/day, about 1×10⁶ cfu to about 1×10⁹ cfu/kg/day, about 1×10⁶ cfu to about 1×10⁸ cfu/kg/day, about 1×10⁶ cfu to about 5×10⁷ cfu/kg/day, or about 1×10⁶ cfu to about 1×10⁷ cfu/kg/day, is contemplated. Preferably, the administration of from about 5×10⁶ cfu to about 5×10⁸ cfu per kg dry feed weight of L. rhamnosus strain FNZ118 per day, preferably about 5×10⁶ cfu to about 4×10⁸ cfu/kg/day, about 5×10⁶ cfu to about 3×10⁸ cfu/kg/day, about 5×10⁶ cfu to about 2×10⁸ cfu/kg/day, about 5×10⁶ cfu to about 1×10⁸ cfu/kg/day, about 5×10⁶ cfu to about 9×10⁷ cfu/kg/day, about 5×10⁶ cfu to about 8×10⁷ cfu/kg/day, about 5×10⁶ cfu to about 7×10⁷ cfu/kg/day, about 5×10⁶ cfu to about 6×10⁷ cfu/kg/day, about 5×10⁶ cfu to about 5×10⁷ cfu/kg/day, about 5×10⁶ cfu to about 4×10⁷ cfu/kg/day, about 5×10⁶ cfu to about 3×10⁷ cfu/kg/day, about 5×10⁶ cfu to about 2×10⁷ cfu/kg/day, or about 5×10⁶ cfu to about 1×10⁷ cfu/kg/day, is contemplated.

In certain embodiments, periodic dose need not vary with body weight, dry feed weight or other characteristics of the subject. In such examples, the administration of from about 1×10⁶ cfu to about 1×10¹³ cfu of L. rhamnosus strain FNZ118 per day, preferably about 1×10⁶ cfu to about 1×10¹² cfu/day, about 1×10⁶ cfu to about 1×10¹¹ cfu/day, about 1×10⁶ cfu to about 1×10¹⁰ cfu/day, about 1×10⁶ cfu to about 1×10⁹ cfu/day, about 1×10⁶ cfu to about 1×10⁸ cfu/day, about 1×10⁶ cfu to about 5×10⁷ cfu/day, or about 1×10⁶ cfu to about 1×10⁷ cfu/day, is contemplated.

In certain embodiments, the administration of from about 5×10⁷ cfu to about 5×10¹⁰ cfu per kg body weight of L. rhamnosus strain FNZ118 per day, preferably about 5×10⁷ cfu to about 4×10¹⁰ cfu/day, about 5×10⁷ cfu to about 3×10¹⁰ cfu/day, about 5×10⁷ cfu to about 2×10¹⁰ cfu/day, about 5×10⁷ cfu to about 1×10¹⁰ cfu/day, about 5×10⁷ cfu to about 9×10⁹ cfu/day, about 5×10⁷ cfu to about 8×10⁹ cfu/day, about 5×10⁷ cfu to about 7×10⁹ cfu/day, about 5×10⁷ cfu to about 6×10⁹ cfu/day, about 5×10⁷ cfu to about 5×10⁹ cfu/day, about 5×10⁷ cfu to about 4×10⁹ cfu/day, about 5×10⁷ cfu to about 3×10⁹ cfu/day, about 5×10⁷ cfu to about 2×10⁹ cfu/day, or about 5×10⁷ cfu to about 1×10⁹ cfu/day, is contemplated. Preferably, a dose of between 1×10⁸ and 1×10⁹ cfu/kg body weight per day is administered.

It will be appreciated that, in certain embodiments, the dose need not be administered daily. For example, the composition may be formulated to be administered every two days, twice weekly, weekly, fortnightly, or monthly. Alternatively, in certain embodiments, the composition may be formulated to be administered 2, 3, 4, 5, 6, 7, 8, 9, 10, 11, 12, 13, 14, 15, 16, 17, 18, 19, 20, 21, 22, 23, or 24 times per day, with every feed, or with every mouthful.

It will be appreciated that the composition is preferably formulated so as to allow the administration of an efficacious dose of L. rhamnosus strain FNZ118 and/or one or more derivatives thereof. The dose of the composition administered, the period of administration, and the general administration regime may differ between animals depending on such variables as mode of administration chosen, and the age, sex, body weight, and species of an animal. Furthermore, as described above the appropriate dose may depend on the nature of the active agent in the composition and the manner of formulation.

Furthermore, the dose of the composition may vary over time. For example, in some embodiments, an initial dosing regimen may be followed by a maintenance dosing regimen. It will be appreciated that a higher dose may be required to establish a population of L. rhamnosus FNZ118 in the animal, and a lower dose may be sufficient to maintain said population. Accordingly, in some embodiments, the initial dosing regimen comprises administering a higher dose and/or a more frequent dose than the maintenance dosing regimen. Preferably, the initial dosing regimen is efficacious to establish a population of L. rhamnosus FNZ118 in the animal, and preferably the maintenance dosing regimen is efficacious to maintain a population of L. rhamnosus FNZ118 in the animal. In some embodiments, the maintenance dosing regimen comprises administering a dose every day, every second day, twice weekly, weekly, fortnightly, or monthly.

In some embodiments, the effect of the methods described herein persist after the administration of the L. rhamnosus FNZ118. Without wishing to be bound by theory, it is anticipated that administration of L. rhamnosus FNZ118 as described herein may result in a long-lasting or even permanent changes in the forestomach and/or rumen of the ruminant animal. In some embodiments, the effect persists for 2 days after the last administration of L. rhamnosus FNZ118, such as 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ118. In a preferred embodiment, the effect persists for the life of the animal.

In examples where the composition comprises one or more derivatives of L. rhamnosus strain FNZ118, the dose may be calculated by reference to the amount or concentration of the derivative to be administered per day. For example, when the bacteria are inactivated, the quantities described previously are calculated before inactivation. For a composition comprising L. rhamnosus strain FNZ118 culture supernatant, the dose may be calculated by reference to the concentration of culture supernatant present in the composition. The concentration of culture supernatant present in the composition may be calculated, for example, on the basis of the cfu of the culture. For example, a dosage of culture supernatant equivalent to 1×10⁹ cfu/day can be calculated from the total yield of the culture and the total volume of the culture supernatant.

It will be appreciated that preferred compositions are formulated to provide an efficacious dose in a convenient form and amount. In certain embodiments, such as but not limited to those where periodic dose need not vary with body weight or other characteristics of the animal, the composition may be formulated for unit dosage. It should be appreciated that administration may include a single daily dose or administration of a number of discrete divided doses as may be appropriate. For example, an efficacious dose of L. rhamnosus strain FNZ118 may be formulated into a feed for oral administration.

However, by way of general example, the inventors contemplate administration of from about 1 mg to about 1000 mg of a composition useful herein per day, preferably about 50 to about 500 mg per day, alternatively about 150 to about 410 mg/day or about 110 to about 310 mg/day. In one embodiment, the inventors contemplate administration of from about 0.05 mg to about 250 mg per kg body weight of a composition useful herein.

In one embodiment a composition useful herein comprises, consists essentially of, or consists of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of L. rhamnosus strain FNZ118 and/or a derivative thereof and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99% from about 30 to about 990%, from about 35 to about 99%, from about 40 to about 99%, and from about 45 to about 99%).

In one embodiment a composition useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of L. rhamnosus strain FNZ118 and/or a derivative thereof and useful ranges may be selected between any of these foregoing values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In certain embodiments, a composition useful herein comprises, consists essentially of, or consists of at least about 10⁴, 10⁵, 10⁶, 10⁷, 10⁸, 10⁹, 10¹⁰, 10¹¹, 10¹², or 10¹³ colony forming units (cfu) of L. rhamnosus strain FNZ118 per kg dry weight of the composition, and useful ranges may be selected between any of these foregoing values (for example, from about 10⁵ to about 10¹³ cfu, from about 10⁶ to about 10¹² cfu, from about 10⁷ to about 10¹² cfu, from about 10⁸ to about 10¹¹ cfu, from about 10⁸ to about 10¹⁰ cfu, and from about 10⁸ to about 10⁹ cfu).

It will be apparent that the concentration of L. rhamnosus strain FNZ118 and/or one or more derivatives thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at an efficacious dose.

The compositions useful herein may be used alone or in combination with one or more other therapeutic agents. The therapeutic agent may be a food, drink, food additive, drink additive, food component, drink component, dietary supplement, vitamin or mineral premix, oil, oil blend, oil rich feed supplement, nutritional product, medical food, nutraceutical, medicament or pharmaceutical. The therapeutic agent may be a probiotic agent or a probiotic factor, and is preferably effective to inhibit the growth of methanogenic bacteria and/or archaea, or to reduce methane emissions by methanogenic bacteria and/or archaea. In some embodiments, the oil, oil blend, or oil rich feed supplement is palm kernel expeller (PKE) and/or PROLIQ.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent are provided. Examples of other therapeutic agents include at least one microorganism of a different species or strain, a vaccine that inhibits methanogens or methanogenesis, and/or a natural or chemically-synthesised inhibitor of methanogenesis and/or methanogen inhibitor, such as bromoform.

Suitable agents with which the compositions useful herein can be separately, simultaneously or sequentially administered include one or more prebiotic agents, one or more probiotic agents, one or more postbiotic agents, one or more phospholipids, one or more gangliosides, other suitable agents known in the art, and combinations thereof.

Typically, the term prebiotic refers to a material that stimulates the growth and/or activity of bacteria in the animals' digestive system that have biologic activity. Prebiotics may be selectively fermented ingredients that allow specific changes, both in the composition and/or activity of the gastrointestinal microflora, which confer health benefits upon the host. Probiotics generally refer to microorganisms that contribute to intestinal microbial balance which in turn play a role in maintaining health, or providing other biologic activity. Many species of lactic acid bacteria (LAB) such as, Lacticaseibacillus and Bifidobacterium are generally considered as probiotics, but some species of Bacillus, and some yeasts have also been found as suitable candidates. Postbiotics refer to non-viable bacterial products or metabolic byproducts from microorganisms such as probiotics, that have biologic activity in the host.

Useful prebiotics include galactooligosaccharides (GOS), short chain GOS, long chain GOS, fructooligosaccharides (FOS), short chain FOS, long chain FOS, inulin, galactans, fructans, lactulose, and any mixture of any two or more thereof. Some prebiotics are reviewed by Boehm G and Moro G (Structural and Functional Aspects of Prebiotics Used in Infant Nutrition, J. Nutr. (2008) 138(9):1818S-1828S), incorporated herein by reference. Other useful agents may include dietary fibre such as a fully or partially insoluble or indigestible dietary fibre.

Accordingly, in one embodiment L. rhamnosus strain FNZ118 and/or a derivative thereof may be administered separately, simultaneously or sequentially with one or more agents selected from one or more probiotics, one or more prebiotics, one or more sources of dietary fibre, one or more galactooligosaccharides, one or more short chain galactooligosaccharides, one or more long chain galactooligosaccharides, one or more fructooligosaccharides, one or more short chain galactooligosaccharides, one or more long chain galactooligosaccharides, inulin, one or more galactans, one or more fructans, lactulose, or any mixture of any two or more thereof.

In certain embodiments, the composition comprises L. rhamnosus strain FNZ118 and/or a derivative thereof and one or more prebiotics, one or more probiotics, one or more postbiotics, one or more sources of dietary fibre. In certain embodiments, the prebiotic comprises one or more fructooligosaccharides, one or more galactooligosaccharides, inulin, one or more galactans, one or more fructans, lactulose, or any mixture of any two or more thereof.

Without wishing to be bound by theory, it is believed that co-culture and/or co-administration of two or more strains of lactic acid bacteria, such as three strains of lactic acid bacteria, can reduce the incidence of culture failure due to infection by bacteriophages. Accordingly, in certain embodiments, the composition comprises L. rhamnosus FNZ118 and one or more other strain of lactic acid bacteria, preferably two or more other strains of lactic acid bacteria. In other embodiments, the composition comprising L. rhamnosus FNZ118 is administered simultaneously or sequentially with one or more other compositions comprising one or more other strains of lactic acid bacteria, preferably two or more other strains of lactic acid bacteria.

It will be appreciated that different compositions of the invention may be formulated with a view to administration to a particular ruminant subject group. For example, the formulation of a composition suitable to be administered to cattle may differ to that suitable to be administered to a different ruminant, such as sheep. It should also be appreciated that compositions of the invention may be formulated differently to be suitable to be administered to ruminant animals of different ages. For example, the formulation of a composition suitable to be administered to calves or lambs may differ to that suitable to be administered to adult cows or sheep. In certain embodiments, a first composition may be formulated for administration to young animals, such as pre-weaning animals, in an initial dosing regimen, and a second composition may be formulated for administration to the same animals in a maintenance dosing regimen. In some embodiments, the first composition is formulated for pre-weaning animals and the second composition is formulated for post-weaning animals.

Preparation of L. rhamnosus Strain FNZ118

Direct-fed microbials (DFMs) and their use in methods to modulate ruminal function and improve ruminant performance is known in the art, as are methods for their production.

Briefly, L. rhamnosus strain FNZ118 can be cultured using conventional liquid or solid fermentation techniques. In at least one embodiment, the strain is grown in a liquid nutrient broth, to a level at which the highest number of cells are formed. The strain is produced by fermenting the bacterial strain, which can be started by scaling-up a seed culture. This involves repeatedly and aseptically transferring the culture to a larger and larger volume to serve as the inoculum for the fermentation, which can be carried out in large stainless steel fermenters in medium containing proteins, carbohydrates, and minerals necessary for optimal growth. Non-limiting exemplary media are MRS or TSB. However, other media can also be used. After the inoculum is added to the fermentation vessel, the temperature and agitation are controlled to allow maximum growth. Once the culture reaches a maximum population density, the culture is harvested by separating the cells from the fermentation medium. This is commonly done by centrifugation.

In one embodiment, to prepare the L. rhamnosus strain FNZ118, the strain is fermented to a 1×10⁸ CFU/ml to about 1×10⁹ CFU/ml level. The bacteria are harvested by centrifugation, and the supernatant is removed. The pelleted bacteria can then be used to produce a DFM. In at least some embodiments, the pelleted bacteria are freeze-dried and then used to form a DFM. However, it is not necessary to freeze-dry the strain before using them. The strain can also be used with or without preservatives, and in concentrated, unconcentrated, or diluted form.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium.

Since multiple cells may give rise to one visible colony, the term colony forming unit is a more useful unit measurement than cell number.

### EXAMPLES

### 1. Example 1—Plate-Based Screen of Bacteriocin Extracts Against Indicator Methanogen Strains

### 1.1 Materials and Methods

### 1.1.1 Bacteriocin Extraction

Bacteriocin extracts from L. rhamnosus FNZ118 cultures were prepared and tested for their effect against indicator methanogen strains Methanobrevibacter boviskoreani JH1 (‘JH1’), Methanosphaera sp. WGK6 (‘WGK6’), Methanobrevibacter ruminantium M1 (‘M1’) and Methanobrevibacter gottschalkii D5 (‘D5’).

L. rhamnosus FNZ118 was revived from −80° C. storage by plating onto De Man-Rogosa-Sharpe agar (MRS, De Man et al., 1960)+lactose (2 g/L). Using a small inoculating loop, glycerol stocks were streaked onto MRS agar plates to obtain an isolated colony. The plates were incubated for 48 h in a sealed container at 37° C. After growth, a single colony was selected, picked up and re-streaked a second time on an agar plate and incubated at 37° C. to obtain an isolated colony. After 48 hours, a single colony from the re-streaked plate was selected and inoculated into the MRS liquid medium, and incubated at 37° C. for 48 h. An inoculum (1 mL) of each revived strain was then sub-cultured into 16 mL MRS+nisin liquid medium (1 ng/mL final conc.). The nisin was included in these media at a very low level to induce bacteriocin production. The cultures were incubated overnight at 37° C. Overnight-grown L. rhamnosus FNZ118 cultures were used for bacteriocin extraction. A drop of the culture was used to make a wet mount slide to examine cells using phase contrast microscopy and to prepare a Gram stain to check culture purity.

The remainder of the culture (˜16 mL) was transferred into a 50 mL Falcon tube and used for bacteriocin extraction following the method of Gaspar et al. (2018), with some modifications as follows. The pH of the culture was adjusted to ˜6.8 with 6M NaOH. Then 0.3 mL of catalase (2 mg/L) was added to the culture and incubated for 30 min at 37° C. followed by an incubation at 70° C. for 45 min. The cultures were then centrifuged for 20 min at 12,000×g at 4° C., the supernatant was decanted, and the cell pellet was resuspended in 8 mL 0.9% NaCl, pH 2. The pH of the resuspended pellet was checked and, if necessary, adjusted to pH 2 with 1M HCl. The cells were incubated for 2 hr at 4° C. with slow agitation on a shaking platform. The cells were then centrifuged at 12,000×g for 20 min at 4° C., and the supernatant collected into a fresh 15 mL Falcon tube. The pH of the supernatant was adjusted to pH 6.8 with 1M NaOH and filtered through a sterile filter (Millex-GP 0.22 μm, 25 mm diameter, Millipore, Merck, Sigma-Aldrich NZ) into a sterile, N2-flushed, Hungate tube using a 10 mL syringe and needle under sterile conditions. The filtered supernatant was frozen at −20° C. until use.

1.1.2 Mbb. boviskoreani JH1 Culture

To identify potential candidate LAB strains with anti-methanogen activities, a microtitre plate-based methanogen growth inhibition bioassay using the model methanogen strain, Methanobrevibacter boviskoreani JH1 (Li et al., 2019) was used. Mbb. boviskoreani JH1 has the unusual ability to grow using ethanol as a source of reducing power to reduce CO₂ to CH₄ allowing JH1 growth in microtitre plates incubated under anaerobic conditions without the need to supply H2 via a 1 atm overpressure of H₂:CO₂ (80:20). This allows a high throughput JH1 screening method to identify inhibitory activities from LAB strains.

The Mbb. boviskoreani JH1 cultures for inoculating the plate assays were grown in Balch tubes (Anaerobic tube, 18×150 mm, butyl rubber septum stopper, aluminium crimps, Bellco Glass, Vineland, NJ, USA) containing 9 mL BY medium (Joblin, 1995) supplemented with (final concentrations) 60 mM sodium formate, 200 mM ethanol, 0.1 mL of Vitamin Solution (1×) and 0.1 mL of Coenzyme M Solution (10 μM) by syringe using anaerobic techniques. The tubes were incubated at 39° C. without shaking until visible turbidity appeared after 3 to 5 days and were used for inoculation of the microtitre plate assays after they attained an OD₆₀₀ of between 0.8 to 1.0 against a distilled water blank. The over-pressure in the JH1 culture tubes was released by inserting a needle through the butyl rubber septum and allowing the accumulated gases to escape, prior to removing the inoculum.

The freshly grown cultures were checked using wet mounts under fluorescence microscopy, and Mbb. boviskoreani JH1 appeared as short ovoid-shaped rods that fluoresced green under ultraviolet (UV) illumination. Culture contamination was checked by inoculating a sample of the culture into 9 mL BY medium supplemented with 5 mM glucose and incubating at 39° C. for one day. If no turbidity was seen after 1 day, then the culture was considered uncontaminated. Further culture verification was conducted from time to time by extracting the genomic DNA from the culture and PCR amplifying the 16S rRNA gene, using both the conventional bacterial 16S primers (27f-GAGTTTGATCMTGGCTCAG, 1492r-GGYTACCTTGTTACGACTT) and the archaeal-specific 16S primers (915af-AGGAATTGGCGGGGGAGCAC, 1386r-GCGGTGTGTGCAAGGAGC). The presence of a band with the archaeal primer set and the absence of a band with the bacterial primer set, and the sequencing results from the PCR products, were used to confirm culture purity.

1.1.3 Methanosphaera sp. WGK6 Culture

Members of the genus Methanosphaera make up around 8% of rumen methanogens (Henderson et al., 2015) and are generally H₂-dependent methylotrophs, using H₂ to reduce methanol to methane. Methanosphaera sp. WGK6 is a H₂-utilising methylotrophic methanogen isolated from the gut of a kangaroo in Australia, but it is also able to use ethanol as a source of reducing power to reduce methanol to methane (Hoedt, 2017). Similar to Mbb. boviskoreani JH1, this metabolic capability theoretically allows WGK6 to grow on ethanol without the need for an over-pressure of H₂, and thus enable it to grow in a microtitre plate. The growth of Methanosphaera sp. WGK6 was tested using BRN-RF10 medium (Balch et al., 1979; Hoedt, 2017) in Hungate tubes with H₂ (180 kPa over-pressure of H₂+CO₂; 80:20) or ethanol as the energy sources and methanol as the terminal electron acceptor in both cases. Attempts to grow WGK6 on ethanol+methanol were unsuccessful, but WGK6 was able to grow on methanol+H₂ in Hungate tubes. Our initial attempts to grow Methanosphaera sp. WGK6 in a microtitre plate format with methanol under a H₂+CO₂ atmosphere (180 kPa over-pressure) in a pressurised gas cannister, produced barely detectable growth after 1 week. However, after increasing the concentration of cysteine added to the BRN-RF10 medium, better growth of Methanosphaera sp. WGK6 was obtained. So, a plate assay using stainless steel gas cannisters able to be pressurize (H₂+CO₂; 80:20) was developed.

The Methanosphaera sp. WGK6 cultures for the assay were grown in Balch tubes in 9 mL BRN-RF10 medium supplemented with (final concentrations) 60 mM sodium formate, 1% methanol, 0.1 mL of Vitamin Solution (1×) and 0.1 mL of Coenzyme M Solution (10 μM) by syringe using anaerobic techniques and with 180 kPa over-pressure of H₂+CO₂ (80:20, BOC Gases NZ). The tubes were incubated at 39° C. without shaking until visible turbidity appeared after 3 to 5 days and were used for inoculation of the tube assays after they attained an OD₆₀₀ of between 0.8 to 1.0 against a distilled water blank. The over-pressure in the WGK6 culture tubes was released by inserting a needle through the butyl rubber septum and allowing the accumulated gases to escape, prior to removing the inoculum.

1.1.4 Mbb. ruminantium M1 and Mbb. gottschalkii D5 Culture

The procedure for growing Methanobrevibacter ruminantium M1 and Methanobrevibacter gottschalkii D5 was identical to the WGK6 protocol described in 1.1.3 above, except it used BY medium for growth. The cultures for the assays were grown in Balch tubes in 9 mL BY medium supplemented with (final concentrations) 60 mM sodium formate, 0.1 mL of Vitamin Solution (1×) and 0.1 mL of Coenzyme M Solution (10 μM) added by syringe using anaerobic techniques and with 180 kPa over-pressure of H₂+CO₂ (80:20, BOC Gases NZ). The tubes were incubated at 39° C. without shaking until visible turbidity appeared after 3 to 5 days and were used for inoculation of the microtitre plate assays.

1.1.5 Mbb. boviskoreani JH1 Growth Inhibition Assay

The bacteriocin extracts from L. rhamnosus FNZ118 stored frozen under anaerobic conditions in Hungate tubes were allowed to thaw at room temperature. All of the assay components for each assay, except the JH1 inoculum, were added via CO₂-flushed syringes and needles to 3.75 mL BY+formate medium in sterile 7.5 mL Hungate tubes in the proportions indicated in Table 1. Each tube was then inoculated with freshly grown JH1 culture, incubated for 1 hr at 39° C., then moved inside an anaerobic chamber (98% CO₂-2% H₂ atmosphere; Coy Laboratory Products, USA) and dispensed into wells of multiwell 96 well plates. The filled plates were placed into an AnaeroPack 2.5 L Rectangular Jar with an MCG Anaeropack-Anaero (Ngaio Diagnostics, Nelson, NZ), the lid sealed, and the jar removed from the anaerobic chamber and incubated at 39° C. The plate was observed daily through the transparent jar, until the Mbb. boviskoreani JH1 control wells showed visible turbidity (usually within 5 to 6 days). The optical density of each well was then recorded at 595 nm (OD₅₉₅) after 5 seconds shaking in a Multiskan FC Microplate Photometer (Thermo Scientific, Auckland, NZ). The absorbance readings of the media control wells were subtracted as background, and the % inhibition of Mbb. boviskoreani JH1 growth caused by the bacteriocin extract samples, relative to the JH1 positive growth control wells (which contained buffer alone) was calculated.

TABLE 1
Microtitre plate setup for the Mbb. boviskoreani
JH1 growth inhibition assay.

| | Amount added (μl) | | | |
|---|---|---|---|---|
| | LAB | | JH1 | |
| | bacteriocin | Media | alone | |
| Component | extract | control | control | Nisin |
| BY medium + | 150 | 150 | 150 | 150 |
| formate (3M) | | | | |
| Phosphate | 5 | 90 | 80 | 70 |
| buffer (1M) | | | | |
| Ethanol (10M) | 5 | 5 | 5 | 5 |
| Vitamin/CoM | 5 | 5 | 5 | 5 |
| solution (1×) | | | | |
| Bacteriocin | 75 | 0 | 0 | 0 |
| extract | | | | |
| Nisin (1 | 0 | 0 | 0 | 10 |
| mg/ml, 300 | | | | |
| μM) | | | | |
| JH1 inoculum | 10 | 0 | 10 | 10 |
| TOTAL | 250 | 250 | 250 | 250 |

1.1.6 Methanosphaera sp. WGK6 Growth Inhibition Assay

Each of the assay components for the assay, except the WGK6 inoculum, were added via C02-flushed syringes and needles to 3.75 mL BRN-RF10 medium in Hungate tubes supplemented with 1% methanol (247 mM, final conc.) as described in Table 2. The tubes were then moved into the chamber, along with the inoculum tube. The medium containing all the components except the inoculum were dispensed into the plates inside the chamber, and then the inoculum was added to the appropriate wells. The plates were placed into a stainless-steel gas cannister laid horizontally, to hold up to 4 microtitre plates at a time. Two anaerobic sachets (MCG Anaeropack-Anaero, Ngaio Diagnostics, Nelson, NZ) were added, the cannister was sealed and the cannister was taken out of the anaerobic chamber and pumped to a pressure 180 kPa with H₂+CO₂ (80:20, BOC Gases NZ), then incubated at 39° C. for 1 week. The cannisters were checked periodically to ensure an over-pressure was maintained, and if necessary, re-pressurised with H₂+CO₂. After incubation for 1 week, the cannister was opened and the plates were removed. The contents of each well were resuspended evenly by repeated pipetting with a multichannel pipettor. The optical density of each well was then immediately recorded at 595 nm (OD₅₉₅) after 5 seconds shaking in a Multiskan FC Microplate Photometer (Thermo Scientific, Auckland, NZ). The absorbance readings of the media control wells were subtracted as background, and the % inhibition of Methanosphaera sp. WGK6 growth caused by the bacteriocin extract samples, relative to the WGK6 positive growth control wells (which contained buffer alone in place of bacteriocin extract) was calculated.

TABLE 2
Microtitre plate setup for the Methanosphaera
sp. WGK6 growth inhibition assay.

| | Amount added (ml) | | | |
|---|---|---|---|---|
| | Media | Nisin | Growth | Bacteriocin |
| Component | control | control | control | test |
| BRN-10 Media | 3.5 | 3.5 | 3.5 | 3.5 |
| 3M Sodium | 0.1 | 0.1 | 0.1 | 0.1 |
| Formate, 1M | | | | |
| Sodium | | | | |
| Acetate, 1M | | | | |
| Methanol mix | | | | |
| 25 mg/ml | 0.1 | 0.1 | 0.1 | 0.1 |
| L-cysteine-HCl | | | | |
| Methanogen | 0 | 0.5 | 0.5 | 0.5 |
| inoculum | | | | |
| Nisin (1 mg/mL, | 0 | 0.2 | 0 | 0 |
| 300 μM) | | | | |
| Bacteriocin | 0 | 0 | 0 | 0.8 |
| extract | | | | |
| 0.9% NaCl | 0.8 | 0.6 | 0.8 | 0 |
| Additional | 0.5 | 0 | 0 | 0 |
| BRN-10 (in | | | | |
| place of inoculum) | | | | |
| TOTAL | 5.0 | 5.0 | 5.0 | 5.0 |

1.1.7 Mbb. ruminantium M1 and Mbb. gottschalkii D5 Growth Inhibition Assays

Cultures of Mbb. ruminantium M1 and Mbb. gottschalkii D5 were prepared as described in 1.1.4 above. The over-pressure in the tubes was released prior to removing the inoculum.

The assay components were added to 3.5 mL of sterile BY medium in a 7.5 mL Hungate tube via CO₂-flushed syringes and needles as describe in Table 3. Each tube was then inoculated with freshly-grown culture, incubated for 1 hr at 39° C., then moved inside the anaerobic chamber and dispensed into wells of 96-well multiwell plates. The plates were sealed and incubated in stainless steel gas cannisters under 180 kPa over-pressure of H₂+CO₂ and their optical densities recorded by spectrophotometric measurement at OD₅₉₅ as described for the Methanosphaera sp. WGK6 assay in 1.1.6 above. The OD₅₉₅ readings of the BY media control wells were subtracted as background, and the % inhibition of the growth of the Mbb. ruminantium M1 or Mbb. gottschalkii D5 caused by bacteriocin extract samples, relative to the positive growth control wells (which contained buffer in place of the bacteriocin extract) were calculated.

TABLE 3
Microtitre plate setup for the Mbb. ruminantium M1
and Mbb. gottschalkii D5 growth inhibition assays.

| | Amount added (ml) | | | |
|---|---|---|---|---|
| | Media | | Growth | Bacteriocin |
| Component | control | Nisin | control | test |
| BY medium | 7.0 | 7.0 | 7.0 | 3.5 |
| 3M sodium | | | | |
| formate/1M | 0.2 | 0.2 | 0.2 | 0.1 |
| sodium | | | | |
| acetate mix/ | | | | |
| 1M methanol | | | | |
| Vitamin/COM | 0.2 | 0.2 | 0.2 | 0.1 |
| solution (1×) | | | | |
| 25mg/ml L- | 0.2 | 0.2 | 0.2 | 0.1 |
| cysteine-HCl | | | | |
| Methanogen | 0 | 1.0 | 1.0 | 0.5 |
| inoculum (M1 | | | | |
| or D5) | | | | |
| Nisin (1 mg/ | 0 | 0.4 | 0 | 0 |
| mL, 300 μM) | | | | |
| Bacteriocin | 0 | 0 | 0 | 0.8 |
| extract | | | | |
| Additional BY | 1.0 | 0 | 0 | 0 |
| medium | | | | |
| NaCl 0.9% | 1.6 | 1.2 | 1.6 | 0 |
| TOTAL | 10.2 | 10.2 | 10.2 | 5.1 |

### 1.2 Results

Bacteriocin extracts from a total of 1,712 strains of lactic acid bacteria were screened against Methanosphaera sp. WGK6. Of these, 1,580 strains (>92%) showed less than 50% inhibition. The 1,712 strains of lactic acid bacteria included 94 strains of Lacticaseibacillus rhamnosus, of which 62 (˜66%) showed less than 20% inhibition of WGK6, 81 (˜86%) showed less than 50% inhibition, and only 3 strains (˜3%) showed ˜80% inhibition or more. Together, this indicates that methanogen inhibition is likely to be a strain-specific effect.

The L. rhamnosus FNZ118 bacteriocin extract showed very strong inhibition of the indicator methylotrophic methanogen Methanosphaera sp. WGK6, but very weak or no inhibition of indicator hydrogenotrophic methanogens Mbb. boviskoreani JH1, Mbb. ruminantium M1, or Mbb. gottschalkii D5, as shown in Table 4.

TABLE 4
Inhibition of indicator methanogen strains by
L. rhamnosus FNZ118 bacteriocin extract.

| | % inhibition | | | |
|---|---|---|---|---|
| | JH1 | WGK6 | M1 | D5 |
| FNZ118 | 1 | 79 | 0 | 0 |

### 1.3 Discussion and Conclusion

Members of the Methanobrevibacter and Methanosphaera genera are the predominant methanogens in the rumen across multiple ruminant species. WGK6 was used as an indicator strain for methylotrophic methanogens in general and Methanosphaera spp. in particular. Mbb. boviskoreani JH1, Mbb. ruminantium M1, and Mbb. gottschalkii D5 were used as indicator strains for Methanobrevibacter spp. This Example shows that L. rhamnosus FNZ118 bacteriocin extract shows a strong inhibitory effect against the methylotrophic methanogen Methanosphaera sp. WGK6, but no effect against the hydrogenotrophic Mbb. boviskoreani JH1, Mbb. ruminantium M1, and Mbb. gottschalkii D5 methanogens.

### 2. Example 2—Impact of L. rhamnosus FNZ118 on Rumen in vitro Assays

### 2.1 Materials and Methods

### 2.1.1 Preparation of Bacterial Cultures and Supernatants for Testing

L. rhamnosus FNZ118 was used to inoculate 7 Hungate tubes, each containing 5 mL of anaerobic MRS medium (Sigma-Aldrich), and these were incubated at 39° C. for 16 hours (until the cultures reached stationary phase). Cultures were pooled into a 250 mL CO₂-flushed serum bottle. An aliquot (1 mL) of the combined cultures was added to 9 mL of sterile MRS medium to measure its OD₆₀₀. Further aliquots (0.5 mL) of the culture mix were inoculated in triplicate into 4.5 mL of sterile anaerobic buffer and serially 10-fold diluted under CO₂ and plated onto MRS plates to determine the number of colony forming units (CFU.mL-1) of original culture. Half of the remaining culture was used for one set of rumen in vitro fermentations (test culture) and the other half was filtered (Millipore 0.22 μm pore size) and the filtrate was placed into a new sterile anaerobic serum bottle (supernatant treatment, SN). Anaerobic phosphate buffer (0.46 M K₂HPO₄; 0.54 M KH₂PO₄, pH 7) was used as the no treatment control (Buffer).

### 2.1.2 Rumen Fluid Preparation and In Vitro Fermentation Set Up

For inoculation of the rumen in vitro fermentation vessels, fresh rumen contents were collected from 6 rumen-fistulated Friesian cows. After squeezing through 1 layer of cheesecloth, the resulting rumen fluids from two animals were combined (approx. 150 mL rumen fluid) giving 3 biological replicates. Aliquots (12.5 mL) of the mixed rumen fluid were added to 0.5 mg dried grass and 36.5 mL of anaerobic phosphate buffer in a 250 mL serum bottle. The treatments (1 mL) of either Buffer, test culture, SN or bacteriocin extract were added before closing the serum bottles with butyl rubber stoppers, giving a final fermentation volume of 50 mL containing 25% rumen fluid (v/v). Gas production and methane content was measured using an automated incubation system (Muetzel et al., 2014).

### 2.1.1 VFA Sample Collections and Analysis

Samples were collected from bottles for VFA analysis. At each time point, 3 mL aliquots were collected, and their pH measured. 1.8 mL samples of these aliquots were used for VFA and non-VFA analyses. VFA samples were centrifuged at 21,000×g for 10 min at 4° C. and 0.9 mL of supernatant was removed and added to 0.1 mL of internal standard (20 mM 2-ethylbutyrate in 20% phosphoric acid), mixed and frozen at −20° C. until analysis. After thawing and re-centrifugation at 21,000×g for 10 min at 4° C., 0.9 mL was collected for derivatization for non-VFA analysis, while the remainder of the sample was analysed directly via GC.

### 2.2 Results

L. rhamnosus FNZ118 was tested for its impact on gas production in rumen in vitro assays, as shown in Tables 5 to 10. The data shown are averages of three replicates. Negative numbers represent stimulation, rather than inhibition. An asterisk (*) is used to indicate statistical significance (* P<0.05; ** P<0.01) by Student's T test with Welch's correction.

TABLE 5
Percent inhibition of total gas produced
(ml per g of substrate) compared to control.

| | RIV | | | | |
|---|---|---|---|---|---|
| | replicate | 2 hours | 6 hours | 12 hours | 20 hours |
| FNZ118 | 1 | 4.2 | 8.8 | 13.2 | 10.1 |
| | 2 | 8.3 | 8.6 | 5.9 | 6.0 |
| | 3 | 2.7 | 1.2 | 1.0 | −0.3 |
| Supernatant | 1 | 20.2 * | 8.2 | 5.8 | 3.7 |
| | 2 | −1.9 | 6.0 | 0.2 | 1.5 |
| Extract | 1 | −3.5 | −3.4 | −2.0 | −3.5 |

TABLE 6
Percent inhibition of total methane produced
(ml per g of substrate) compared to control.

| | RIV | | | | |
|---|---|---|---|---|---|
| | replicate | 2 hours | 6 hours | 12 hours | 20 hours |
| FNZ118 | 1 | 45.1 * | 22.2 * | 15.6 | 8.9 |
| | 2 | 20.5 * | 21.2 ** | 9.1 | 6.8 |
| | 3 | 10.7 * | 5.5 | 2.2 | 1.4 |
| Supernatant | 1 | 36.2 ** | 11.2 ** | 5.9 | 3.0 |
| | 2 | −3.1 | 3.8 | −2.8 | −0.1 |
| Extract | 1 | 15.4 | 5.0 | 2.5 | −0.4 |

TABLE 7
Percent inhibition of total volatile fatty acids
produced (mM) compared to control.

| | RIV | | | | |
|---|---|---|---|---|---|
| | replicate | 0 hours | 6 hours | 12 hours | 24 hours |
| FNZ118 | 1 | 17.3 | 4.1 | −17.0 | 28.3 |
| | 3 | 1.7 | −0.7 | −4.1 | 4.0 |
| Supernatant | 1 | −3.6 | 2.6 | −15.1 | 0.1 |
| Extract | 1 | 0.6 | −7.7 | 8.5 | 6.1 |

TABLE 8
Percent inhibition of acetic acid produced (mM) compared to control.

| | RIV | | | | |
|---|---|---|---|---|---|
| | replicate | 2 hours | 6 hours | 12 hours | 20 hours |
| FNZ118 | 1 | 23.3 | 6.7 * | −18.5 | 27.5 |
| | 3 | 1.6 | −0.3 | −4.1 | 4.0 |
| Supernatant | 1 | −4.5 | 2.4 | −16.6 | −0.6 |
| Extract | 1 | 3.5 | −13.8 | 5.4 | 6.7 |

TABLE 9
Percent inhibition of propionic acid produced (mM) compared to control.

| | RIV | | | | |
|---|---|---|---|---|---|
| | replicate | 2 hours | 6 hours | 12 hours | 20 hours |
| FNZ118 | 1 | 20.4 | 5.9 | −16.3 | 28.3 |
| | 3 | 0.8 | −3.1 | −4.7 | 3.6 |
| Supernatant | 1 | −7.2 | −0.7 | −14.0 | 0.5 |
| Extract | 1 | 2.5 | −13.5 | 7.9 | 6.1 |

TABLE 10
Percent inhibition of butyric acid produced (mM) compared to control.

| | RIV | | | | |
|---|---|---|---|---|---|
| | replicate | 2 hours | 6 hours | 12 hours | 20 hours |
| FNZ118 | 1 | 19.6 | 10.5 | −11.2 | 33.4 |
| | 3 | 2.6 | 1.1 | −3.6 | 3.7 |
| Supernatant | 1 | −5.8 | 1.5 | −10.7 | 4.0 |
| Extract | 1 | 0.9 | −11.8 | 5.0 | 5.1 |

L. rhamnosus FNZ118 produced a significant decrease in total methane produced at 2 and 6 hours in the rumen in vitro assays, across three biological replicates. This effect was also seen using culture supernatant in one of the biological replicates. The bacteriocin extract showed no significant effect. It should be noted that the rumen in vitro assays are a closed system and may become nutrient-limited over time. Therefore, the 0-12 hour timepoints may more accurately reflect the situation in vivo, as animals will typically ingest more food and liquid over a 24-hour period. It should also be noted that the RIV replicates were undertaken at different times, using rumen fluid taken from different cows on a pasture-based diet. The variability between RIV replicates may therefore at least partially be attributable to seasonal changes in pasture quality.

Overall, L. rhamnosus FNZ118 culture, culture supernatant, and bacteriocin extract showed no significant effect on the total gas produced in the rumen in vitro assays, except for one of the biological replicates using culture supernatant.

There was also no significant effect on total volatile fatty acids produced, or the amount of acetic, propionic, and butyric acids produced, except for one of the biological replicates using FNZ118 culture at 6 hours.

### 2.3 Conclusion

The rumen in vitro assay of the L. rhamnosus FNZ118 demonstrated impacts on fermentation end products, showing a significant decrease in methane production. This occurred without significantly affecting volatile fatty acid production.

### 3. Example 3—Calf Methane Emissions

### 3.1 Materials and Methods

### 3.1.1 Calf Study Design, Animal Ethics and Rearing Facility

This Example used a design involving treatment with L. rhamnosus FNZ118 and a control to test the ability of FNZ118 to reduce CH₄ emissions from calves when fed throughout their first 14 weeks of life. Statistical power calculations using data from previous CH₄ emission measurements of calves indicated that at least 20 animals per treatment group were needed to detect a 20% difference in CH₄ emissions. Previous studies have experienced calf exclusions from the trials due to navel infections caused by navel sucking by pen mates. To mitigate against these potential losses 24 calves/group were used. This work was approved by the AgResearch Ruakura Animal Ethics Committee. The calf rearing shed was divided into pens, each approximately 12 m². Each pen accommodated 4 calves and was bedded with woodchips, and was supplied with fresh water and fitted with feeders for calf pellets and hay.

### 3.1.2 Calf Enrolment and Feeding

Female Friesian dairy calves were enrolled into the study over a 3 week period. Calf enrolment was staggered over a 3 week period to spread the calf age such that three groups of similarly-aged animals could be measured sequentially through the cattle CH₄ measurement chambers. Newly-born calves were collected from their mothers twice daily and taken to the calf rearing shed. The calves were weighed on arrival at the shed and then weekly during their time in the calf rearing facility. The calves were assigned to the treatment group (FNZ118 or control) at random within each week of enrolment with balancing of birth weight and calf sire, such that 8 calves were assigned to each treatment group per week. Within the first 12 hr of entering the shed, each calf was offered 2 feeds of 2-3 L of warm colostrum, with the first morning colostrum feed containing the FNZ118 or Control treatments. Freeze-dried L. rhamnosus FNZ118 was kept at −20° C. until use. Each FNZ118-treated calf received a daily dose of FNZ118 of 5×10¹⁰ CFU. The Control treatment (3 g maltrin/calf/d) was the excipient used for blending the concentrated freeze-dried L. rhamnosus FNZ118 product to the correct daily dose. After colostrum feeding, the calves were fed 6 L of calf Milk Replacer (CMR) (Ancalf, NZAgBiz/Fonterra; 150 g/L mixed with tap water at ˜37° C.) daily, divided into 3 L morning and 3 L afternoon feedings. The FNZ118 and Control treatments were added to the morning 3 L milk only and were mixed into the prewarmed CMR until the freeze-dried material was evenly distributed throughout the milk. The calves were also offered solid feed in the form of a pelleted calf feed containing 20% fibre source (lucerne and soy hulls) and a coccidia-specific, non-ionophore coccidiostat (see Table 11). No hay was offered to calves during the first 6 weeks to avoid likely variation in solid feed consumption between calves, and the possible impact of this on rumen development and potentially on calf CH₄ emissions. However, meadow hay and chaffed meadow hay (cut to ˜75 mm) were offered to the calves after their first CH₄ measurements at Week 6 to help stimulate salivary secretions and stabilise rumen pH. The intake of pelleted feed and hay was measured at the pen level.

The calves were weaned off most of their milk starting in Week 10 so that by the end of week 11 they were receiving only 0.5 L of CMR in the morning containing the FNZ118 and Control treatments. The treatments continued to be delivered to calves in 0.5 L CMR in the morning feed until after completion of their second round of CH₄ measurements at 14 weeks.

TABLE 11
Ingredient and nutrient composition of pelleted calf feed.

| Ingredient | Inclusion rate (kg) |
|---|---|
| Maize | 240 |
| Soya 48 | 200 |
| Barley | 100 |
| Wheat | 80 |
| Molasses cane | 50 |
| Lucerne dried | 150 |
| Soya hulls | 150 |
| Limestone | 15 |
| Dicalcium phosphate | 10 |
| Salt | 10 |
| Calf premix + Coccistop (Decoquinate, | 2 |
| ethyl-6-decyloxy-7-ethoxy-4- | |
| hydroxyquinoline-3-carboxylate ) | |
| Mycofix | 1 |
| Total | 1008 |
| ME (MJ/kg) | 12 |
| Crude protein | 20.3 |
| Neutral detergent fibre | 24.3 |
| Starch | 29.5 |
| Sugar | 7 |
| Crude ash | 8.8 |
| Calcium | 1.3 |
| Phosphorus | 0.52 |

### 3.1.3 Animal Health

Calves were disbudded, received vaccinations and anti-parasitic treatments according to animal health and welfare protocols.

### 3.1.4 Calf Methane Emissions Measurements

CH₄ measurements were carried out on 20 calves per treatment group at 6 weeks of age (pre-weaning) and on the same set of calves after 14 weeks of age (post-weaning). The calves were measured in 4 cattle respiration chambers, 4 calves at a time for 2 days each. The first, pre-weaning period of CH₄ measurements occurred over a 34 d period and the second, post-weaning period was also over 34 d.

### First Round CH₄ Measurements

Calves (4 per trip) were transported from the calf rearing shed to the methane measurement facility located at the New Zealand Animal Ruminant Methane Measurement Centre. On arrival, the calves were housed in pens with wood chip bedding and received their afternoon allowance of milk (3 L, pre-weaning) or solid feed (post-weaning) and had fresh water available. The following morning at ˜8:00 am, the calves were moved into individual cattle respiration chambers where they received their morning allowance of milk (CMR; 3 L), and had pelleted feed (plus chaffed hay post-weaning) and water available ad libitum. Calves continued to receive their FNZ118 and Control treatments and solid pelleted feed while in the chambers. The calves remained in the chambers for 2 days of CH₄ measurements and feeding and cleaning of the pens occurred twice daily. At 8:00 am on the third day, the calves were moved from the chambers to a pen and the next 4 calves (kept in a pen over the previous night) entered the chambers. At ˜ 9:00 am the calves exiting the chambers were given their morning allocation of milk and ad libitum pellets (plus chaffed hay post-weaning) and water was freely available. Two hours after feeding (˜11 am), each calf had samples of rumen contents and faecal material collected. The samples of rumen (via stomach tubing) and faecal material (via digital collection) were used immediately for pH measurements and stored at −80° C. for subsequent volatile fatty acid (VFA) analysis.

After the first CH₄ measurement period at 6 weeks, the calves were returned to the calf rearing facility and hay was offered ad libitum in addition to the ad libitum calf pellets until Week 12.

### Second Round CH₄ Measurements

For the 2 weeks prior to the second round of CH₄ measurements at Week 14, the hay was replaced by chaffed hay ad libitum. When the calves entered the respiration chambers for the post-weaning measurement at 14 weeks of age, chaffed hay was offered at ˜10% (500 g/d) of the daily calf pellets offered (5 kg). Intakes of milk and solid feed were measured in both rounds of CH₄ measurements, and samples of the feed were dried to estimate the dry matter intake (DMI) per calf per day and for compositional analysis. Chamber measurements of CH₄, hydrogen (H₂), and CO₂ were reported as emissions (g/d) or as yields (g/kg DMI/d).

After completion of the second round of CH₄ measurements and samplings at week 14, the calves were transported to farms for adaptation to pasture feeding. The calves were placed on pasture with calf pellets available at their previous daily intake and the quantity of pellets offered was reduced over a 3-week period to encourage the transition to pasture (33 percent reduction in pellet allowance per week).

### 3.1.5 Animal Growth Post-Weaning

After the methane measurements at week 14, the calves were moved to pastures and their weight and average daily gains were monitored monthly through to 13 months of age.

Treated and control animals were moved to separate pastures, and pasture quality was monitored to ensure comparability of the results. While there was seasonal variation in metabolisable energy (ME) and digestibility (in vitro DOMD %) in both pastures, as expected in a pasture-based farming system, there was no significant difference between pastures. Treatment with FNZ118 stopped after weaning at 14 weeks of age.

Statistical analysis was performed in R using the Ime4 package. The model for body weight was fitted by REML and includes the effect of treatment with FNZ118, time (i.e. month of weighing), and the interaction of the two, with the random effects of the animal.

### 3.2 Results

A total of 48 female Friesian calves were enrolled into the study over a 3-week period from the 23^{rd} July to the 12^{th} August 2021. As calves were born, they were allocated to one of the 2 treatment groups (n=24 per group)—FNZ118 or Control (excipient only). 20 calves from each group were used for the measurements described below.

### 3.2.1 Animal Growth Pre-Weaning

The calves were weighed on their first arrival at the calf rearing shed. There was no statistical difference in birth weight between the treatment groups (Table 12).

TABLE 12
Calf birth weights.

| Treatment group | Birth weight (kg) | Comparison |
|---|---|---|
| FNZ118 | 36.64 | No significant |
| Control | 35.63 | difference (p = 0.35) |

The calves received their treatments from their first feed of colostrum and continued receiving the treatments once daily in the morning until they completed their second CH₄ measurement after 14 weeks of age. The intake of calf pellets and hay whilst calves were in respiration chambers during methane measurements is shown in Table 13.

TABLE 13
Dietary intake* of calves during CH₄ measurements at 6
and 14 weeks of age.

| | Treatment | Week 6 | | | | Week 14 | | | |
|---|---|---|---|---|---|---|---|---|---|
| Diet | (n = 6) | Mean | SD | p | Sig | Mean | SD | p | Sig |
| Pellets | FNZ118 | 0.683 | 0.095 | 0.99 | NS | 3.825 | 0.28 | 0.5 | NS |
| | Control | 0.682 | 0.126 | na | na | 4.029 | 0.656 | na | na |
| Hay | FNZ118 | na | na | na | na | 0.352 | 0.076 | 0.65 | NS |
| | Control | na | na | na | na | 0.33 | 0.087 | na | na |

*During methane measurements, all calves consumed 6 L of CMR (in 2 × 3 L feeds am and pm) each day at 6 weeks of age, and 0.5 L of CMR each day in the morning only at 14 weeks of age. Hay was not offered to calves until after completion of the first CH₄ measurement at 6 weeks of age.
NS, not significantly different from control by T-test;
na, not applicable.

A summary of the liveweights (LWT) and average daily gains (ADG) prior to the CH₄ measurements at 6 weeks and 14 weeks are shown in Table 14. There were no significant differences in LWT or ADG between the FNZ118 treatment and Control calves at 6 or 14 weeks of age.

It is known that CH₄ measurements in respiration chambers may cause stress to animals, which can affect feed intake and growth rates, therefore, the LWT and ADG of the calves while in the respiration chambers were also examined. A few animals lost weight while going through the chambers (mean LWT loss 0.392 kg over 4 days), but the majority of animals maintained or gained weight (mean LWT gain 0.774 kg over 4 days, ADG 0.194 kg). While in the chambers there were no significant differences in ADGs between the FNZ118 treatment calves compared to the calves fed the Control treatment.

TABLE 14
Dietary intake* of calves during CH₄ measurements at 6
and 14 weeks of age.

| | Treatment | Week 6 | | | | Week 14 | | | |
|---|---|---|---|---|---|---|---|---|---|
| Diet | (n = 20) | Mean | SD | p | Sig | Mean | SD | p | Sig |
| Pellets | FNZ118 | 72.47 | 6.63 | 0.63 | NS | 124.25 | 12.22 | 0.25 | NS |
| | Control | 73.3 | 3.86 | | | 127.93 | 7.08 | | |
| Hay | FNZ118 | 0.88 | 0.24 | 0.72 | NS | 1.03 | 0.31 | 0.73 | NS |
| | Control | 0.9 | 0.15 | | | 0.99 | 0.34 | | |

SD: standard deviation;
p: T-test probability of FNZ118 treatment vs Control,
Sig: statistical significance;
NS, no significant.

### 3.2.2 Methane Emissions

From the calves enrolled into the study, 40 calves were selected for measurement of CH₄ emissions. The selections were made from the weekly enrolment of a cohort of animals and the criteria used for calf exclusions were previous health status and whether there were any veterinary interventions needed prior to CH₄ measurements. The animals were measured in two rounds; Round 1 was pre-weaning at 6 weeks of age and Round 2 was post-weaning at 14 weeks of age. Each round of measurements was carried out in 3 batches over a 34 d period.

In Round 1 the calves received 2×3 L/d of CMR in the morning (containing the treatments) and afternoon feeds, plus calf pellets ad libitum during their time in the chambers. In Round 2 the calves received only 0.5 L of CMR containing their treatments in the morning feed plus ad libitum calf pellets with 10% of their anticipated solid feed intake as chaffed hay also offered.

TABLE 15
Calf CH₄ emissions at 6 weeks (pre-warning) and 14 weeks,
9 months, and 1 year of age (post-weaning).

| | | | CH₄ | | H₂ | | CO₂ | |
|---|---|---|---|---|---|---|---|---|
| CH₄ chamber | Treatment | DMI | | (g/kg | | (g/kg | | (g/kg |
| period | (n = 20) | (kg/d) | (g/d) | DMI) | (g/d) | DMI) | (g/d) | DMI) |
| Week 6 | FNZ118 | 0.61* | 7.51*** | 12.59* | 0.05** | 0.08* | 1633.16*** | 2844.98^{NS} |
| | Control | 0.72 | 10.24 | 14.89 | 0.09 | 0.14 | 1791.52 | 2686.32 |
| Week 14 | FNZ118 | 3.07** | 40.86*** | 13.83^{NS} | 0.14^{NS} | 0.04^{NS} | 2894.25*** | 987.24^{NS} |
| | Control | 3.68 | 52.28 | 14.6 | 0.16 | 0.05 | 3338.65 | 938.39 |
| 9 months | FNZ118 | 6.28 | 101.58*** | 16.26** | 0.28* | 0.05 | 4721.87** | 756.95 |
| | Control | 6.54 | 116.78 | 18.06 | 0.39 | 0.06 | 5081.22 | 783.96 |
| 1 year | FNZ118 | 5.28*** | 131.28*** | 25.39 | 0.10*** | 0.02* | 5842.62*** | 1139.29*** |
| | Control | 6.80 | 158.00 | 23.41 | 0.29 | 0.04 | 6564.41 | 973.95 |

DMI: Dry matter intake. For the first round of measurements at Week 6, calves consumed 6 L CMR/d (900 g/d milk solids) plus the indicated amount of calf pellets, which represented ~40% of total intake. For the second round at Week 14, calves consumed 0.5 L CMR and were offered pellets (5 kg/d) with 10% of anticipated intake offered as chaffed hay (500 g/d). During the 9 month and 1 year measurements, animals were fed cut pressure.
*P < 0.05;
**P < 0.01;
***P < 0.001.

The CH₄ production from calves in Week 6 measurements ranged from 7-10 g/d and the CH₄ yields from 12-15 g/kg DMI/d (Table 15). This is expected from pre-weaned calves on a mainly milk diet (6 L/d: 0.9 kg milk solids) with only a small (0.6-0.7 kg/d) solid feed intake.

The calves receiving the FNZ118 treatment showed a significant (P<0.001) 27% reduction in CH₄ production (g/d) and a significant (P<0.05) 15% reduction in CH₄ yield (g/kg DMI) compared to the calves receiving the Control Treatment (excipient only).

Similarly, there was a significant (P<0.01) 44% reduction in H₂ production (g/d) and CO₂ production (P<0.001) and a significant (P<0.05) 43% reduction in H₂ yield (g/kg DMI) compared to the control calves. The calves receiving the FNZ118 treatment had significantly (P<0.05) lower intake of pellets by ˜15%. However, the calves also consumed 6 L CMR/d (900 g/d milk solids) so the pellet intake represented only ˜40% of the total intake.

After weaning at week 14, CH₄ production was 40.86 g/d for calves treated with FNZ118, and 52.28 g/d for control calves, while CH₄ yield was 13.83 g CH₄/kg DMI/d for FNZ118 treated calves and 14.6 g CH₄/kg DMI/d for control calves. These levels of CH₄ emissions are less than adult animals but are in the range expected for weaner calves. FNZ118-fed calves continued to have lower CH₄ production (g CH₄/day) compared to the Control calves (approx. 22% lower; P<0.001). The CH₄ yield (g CH₄/kg DMI/day) of the FNZ118-fed calves was numerically lower than control (˜5%) but not significantly different from Control calves. CO₂ production was significantly lower in the FNZ118-fed calves (P<0.001) but CO₂ yield, H₂ production and H₂ yield were not different from the Control animals. The FNZ118-fed calves also had a significantly lower (˜17%) DMI (P<0.01).

FNZ118-fed animals continued to have significantly lower CH₄ production (g CH₄/day) at nine months and one year of age, even though supplementation with FNZ118 ceased after the week 14 methane measurements (P<0.001; Table 15). The FNZ118-fed animals also showed lower total dry matter intake than control animals (Table 15). While this did not rise to the level of significance at nine months, the difference was highly significant at one year (P<0.001).

Samples of rumen contents and faecal material were collected from calves after the week 6, week 14, 9 months, and 1 year CH₄ measurement rounds, and their pH values were measured. There were no significant differences between the FNZ118 treatment and Control groups (Table 16).

TABLE 16
Rumen and faecal pH measurements.

| CH₄ | | | | | | | | | |
|---|---|---|---|---|---|---|---|---|---|
| chamber | Treatment | Rumen | | | | Faecal | | | |
| period | (n = 20) | Mean | SD | p | Sig | Mean | SD | p | Sig |
| Week 6 | FNZ118 | 5.86 | 0.54 | 0.59 | NS | 7.25 | 0.66 | 0.69 | NS |
| | Control | 5.96 | 0.58 | | | 7.33 | 0.65 | | |
| Week 14 | FNZ118 | 5.99 | 0.54 | 0.07 | NS | 7.53 | 0.63 | 0.55 | NS |
| | Control | 6.32 | 0.58 | | | 7.65 | 0.63 | | |
| 9 months | FNZ118 | 7.25 | 0.57 | 0.66 | NS | 7.21 | 0.54 | 0.75 | NS |
| | Control | 7.31 | 0.22 | | | 7.15 | 0.63 | | |
| 1 year | FNZ118 | 7.72 | 0.39 | 0.61 | NS | 7.43 | 0.59 | 0.07 | NS |
| | Control | 7.65 | 0.45 | | | 7.13 | 0.37 | | |

Volatile fatty acids in rumen contents were measured from samples collected from the calves after exiting the chambers at Week 6 (pre-weaning) and Week 14 (post-weaning). Acetate was the main VFA detected, while propionate and butyrate made up smaller proportions (Table 17). There were no significant differences between the amount of acetate, propionate, butyrate or total VFAs found in calves fed FNZ118 compared to the Control at Week 6 or Week 14. At 9 months and 1 year, there was a significant decrease in acetate and butyrate, as well as total VFAs in the treated animals, but the acetate:propionate ratio remained the same compared to the control. There was also a decrease in propionate that did not reach significance at 9 months, but was significant at 1 year.

TABLE 17
Volatile fatty acids* in calf rumen samples

| CH₄ chamber | | | | | | | | | |
|---|---|---|---|---|---|---|---|---|---|
| period | | | | | | | | | |
| Treatment | | Week 6 | | Week 14 | | 9 months | | 1 year | |
| (n = 20) | | FNZ118 | Control | FNZ118 | Control | FNZ118 | Control | FNZ118 | Control |
| Acetate | Mean | 52.1 | 52.6 | 63.9 | 57 | 31.7 | 37.4 | 28.9 | 40.8 |
| | SD | 12.9 | 10.6 | 22.1 | 19.6 | 7.3 | 10.4 | 12.5 | 16.7 |
| | p | 0.885 | | 0.318 | | 0.049 | | 0.017 | |
| | Sig | NS | | NS | | * | | * | |
| Propionate | Mean | 39.6 | 37.2 | 31.7 | 28.1 | 8.1 | 9.4 | 7.0 | 9.8 |
| | SD | 11.1 | 8.6 | 9.6 | 13.8 | 1.9 | 2.8 | 3.1 | 4.1 |
| | p | 0.455 | | 0.366 | | 0.089 | | 0.022 | |
| | Sig | NS | | NS | | NS | | * | |
| Butyrate | Mean | 6.9 | 8.5 | 9.1 | 8.7 | 3.8 | 5.4 | 3.4 | 5.5 |
| | SD | 3.5 | 3 | 3.5 | 4.1 | 1.0 | 1.6 | 1.6 | 2.2 |
| | p | 0.156 | | 0.77 | | 0.0009 | | 0.002 | |
| | Sig | NS | | NS | | *** | | ** | |
| Total VFAs | Mean | 104.9 | 104.8 | 111.7 | 1101 | 45.9 | 54.7 | 41.3 | 58.3 |
| | SD | 26.6 | 18 | 35 | 38.5 | 10.4 | 14.9 | 17.8 | 23.4 |
| | p | 0.992 | | 0.383 | | 0.042 | | 0.015 | |
| | Sig | NS | | NS | | * | | * | |

*Values are expressed as mM of individual VFAs as determined by the GC method.
p: T-test probability;
NS: no significantly different from control;
*p < 0.05;
**p < 0.01;
***p < 0.001.

There was a significant increase in the amount of iso-butyrate in treated animals compared to control animals at 6 weeks, 9 months, and 1 year; and a significant increase in the amount of iso-valerate at 9 months and 1 year (Table 18).

TABLE 18
Proportions of minor volatile fatty acids in calf rumen samples.

| CH₄ chamber | | | | | | | | | |
|---|---|---|---|---|---|---|---|---|---|
| period | | | | | | | | | |
| Treatment | | Week 6 | | Week 14 | | 9 months | | 1 year | |
| (n = 20) | | FNZ118 | Control | FNZ118 | Control | FNZ118 | Control | FNZ118 | Control |
| Iso-butyrate | Mean | 0.6 | 0.5 | 0.4 | 0.4 | 1.7 | 1.4 | 1.7 | 1.4 |
| | SD | 0.3 | 0.1 | 0.2 | 0.1 | 0.2 | 0.1 | 0.3 | 0.4 |
| | p | 0.032 | | 0.201 | | 2.63e−5 | | 0.023 | |
| | Sig | * | | NS | | *** | | * | |
| Valerate | Mean | 3.1 | 2.8 | 3.0 | 3.4 | 0.9 | 0.9 | 0.8 | 0.8 |
| | SD | 1.6 | 1.0 | 2.2 | 1.5 | 0.1 | 0.1 | 0.1 | 0.1 |
| | p | 0.453 | | 0.498 | | 0.235 | | 0.326 | |
| | Sig | NS | | NS | | NS | | NS | |
| Iso-valerate | Mean | 0.8 | 0.6 | 0.3 | 0.4 | 2.2 | 2.0 | 1.9 | 1.7 |
| | SD | 0.4 | 0.2 | 0.2 | 0.1 | 0.3 | 0.2 | 0.3 | 0.4 |
| | p | 0.069 | | 0.691 | | 0.002 | | 0.039 | |
| | Sig | NS | | NS | | ** | | * | |
| Caproate | Mean | 0.4 | 0.4 | 0.0 | 0.0 | 0.3 | 0.3 | 0.2 | 0.2 |
| | SD | 0.2 | 0.4 | 0.0 | 0.0 | 0.0 | 0.0 | 0.06 | 0.05 |
| | p | 0.622 | | na | | 0.716 | | 0.097 | |
| | Sig | NS | | NS | | NS | | NS | |

Values are expressed as % of individuals VFAs relative to total VFAs.
p: T-test probability;
NS: not significantly different from control;
*p < 0.05;
**p <0.01;
***p < 0.001.

There were no significant differences in the amount of formate, lactate, and succinate found in treated or control animals at 6 and 14 weeks (Table 19). At 9 months and 1 year, there was a significant decrease in the amount of succinate in treated animals compared to control animals; and there was also a significant decrease in the amount of lactate at 1 year.

TABLE 19
Proportions of minor volatile fatty acids in calf rumen samples.

| CH₄ chamber | | | | | | | | | |
|---|---|---|---|---|---|---|---|---|---|
| period | | | | | | | | | |
| Treatment | | Week 6 | | Week 14 | | 9 months | | 1 year | |
| (n = 20) | | FNZ118 | Control | FNZ118 | Control | FNZ118 | Control | FNZ118 | Control |
| Formate | Mean | 0.000 | 0.009 | 0.053 | 0.112 | 0.031 | 0.065 | 0.04 | 0.06 |
| | SD | 0.000 | 0.029 | 0.111 | 0.202 | 0.036 | 0.091 | 0.08 | 0.08 |
| | p | na | | 0.261 | | 0.123 | | 0.43 | |
| | Sig | na | | NS | | NS | | NS | |
| Lactate | Mean | 2.728 | 2.829 | 2.56 | 2.57 | 0.206 | 0.195 | 0.03 | 0.14 |
| | SD | 2.484 | 2.614 | 4.04 | 3.37 | 0.067 | 0.030 | 0.1 | 0.17 |
| | p | 0.722 | | 0.989 | | 0.507 | | 0.019 | |
| | Sig | NS | | NS | | NS | | * | |
| Succinate | Mean | 0.000 | 0.021 | 0.252 | 0.690 | 0.131 | 0.175 | 0.07 | 0.17 |
| | SD | 0.000 | 0.055 | 0.171 | 1.359 | 0.038 | 0.067 | 0.1 | 0.13 |
| | p | na | | 0.160 | | 0.013 | | 0.015 | |
| | Sig | na | | NS | | * | | * | |

Values are expressed as mM of individual VFAs as determined by the VFA derivatisation.
p: T-test probability;
NS: not significantly different from control;
*p < 0.05;

### 3.2.3 Animal Growth Post-Weaning

After the methane measurements at week 14, the calves were moved to pastures and their weight and average daily gains were monitored monthly.

At 9 months, the FNZ118-treated animals showed numerically lower dietary intake, although this did not reach the level of significance. At 1 year, the treated animals showed significantly lower dietary intake (Table 20).

TABLE 20
Dietary intake* of animals during CH₄ measurements at
9 months and 1 year of age.

| | | 9 months | | | | 1 year | | | |
|---|---|---|---|---|---|---|---|---|---|
| Diet | Treatment | Mean | SD | p | Sig | Mean | SD | p | Sig |
| Pasture | FNZ118 | 6.28 | 0.56 | 0.11 | NS | 5.28 | 1.06 | <0.001 | *** |
| | Control | 6.54 | 0.78 | na | na | 6.80 | 0.95 | na | na |

*Values are weights of pasture dry matter (kg) per animal per day while in the methane measurements chambers.
p: T-test probability;
NS: not significantly different from control;
***p < 0.001.

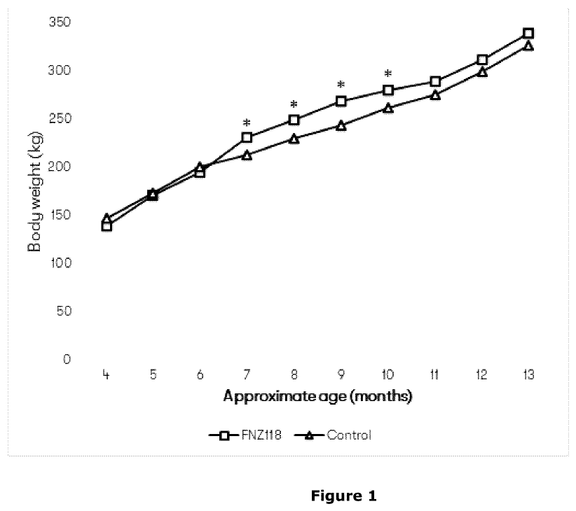

The body weights of the FNZ118-treated and control animals was similar (p>0.05) from 4 months to 6 months (Table 21; FIG. 1). However, from month 7 to month 10 the FNZ118-treated animals showed significantly more gain in body weight, and consequently their body weight was significantly higher (p<0.05), than the control animals (Table 21; FIG. 1). From month 11 onward, the FNZ118-treated animals still showed numerically higher body weight than control animals, although this did not reach the level of significance. Treated animals also showed significantly higher (p<0.001) average daily gain (ADG) than control animals over the entire month 4 to month 13 period.

TABLE 21
Mean monthly body weight and average
daily gain post-weaning.

| Approximate | Body weight (kg) | |
|---|---|---|
| age (months) | FNZ118 | Control |
| 4 | 139 | 146 |
| 5 | 170 | 173 |
| 6 | 195 | 200 |
| 7 | 231* | 213 |
| 8 | 249* | 230 |
| 9 | 269* | 244 |
| 10 | 280* | 262 |
| 11 | 290 | 276 |
| 12 | 312 | 300 |
| 13 | 339 | 326 |
| | Average daily gain (kg/day) | |
| | 0.7141*** | 0.6481 |

*p < 0.05;
***p < 0.001.

### 3.3 Discussion

The Round 1 CH₄ measurements (week 6) showed that calves receiving the FNZ118 Treatment had approximately 27% lower CH₄ production and 15% lower CH₄ yield. Overall, the CH₄ production and CH₄ yield in these young calves were low compared to mature cattle on pasture diets (typically ˜22 g/kg DMI). This was expected from pre-weaned calves on a mainly milk diet (6 L/d: 0.9 kg milk solids) consuming a small amount (0.6-0.7 kg/d) of solid (grain-based) feed. The corresponding strong reduction in H₂ and CO₂ production (P<0.001) and a lower H₂ yield in FNZ118-fed calves were consistent with the low CH₄ production and yields.

The feeding of the FNZ118 strain did not affect CMR intake or the consumption of the solid feed in pens during the period leading up to the first CH₄ measurement. While the calves consumed all of their CMR during Round 1 CH₄ measurements, the intake of the pellets in the FNZ118-fed calves while in the chambers was approximately 100 g/d lower than in the calves fed the control diet, representing ˜15% lower solid feed intake. The stress of CH₄ measurements in respiration chambers may cause some animals to lose weight but the majority of calves in Round 1 continued to gain weight, albeit at a lower rate compared to when the animals were not in chambers. There were no significant differences in ADGs between the calves fed FNZ118 compared to the Control treatment while the animals were in the chambers, so the differences observed in solid feed intake while in the chambers appears to be an effect of the LAB treatment. In these pre-weaning calves, the milk component made up the majority (˜60%) of the dietary intake, so the lower pellet intake did not affect the overall group LWTs or ADGs of the calves receiving the FNZ118 treatment.

After weaning at Week 14, the CH₄ production measured from the calves increased by around 5-fold while their CH₄ yields remained similar to the Week 6 measurements. The increase in overall CH₄ production was expected as the post-weaning diet consisted mainly of pellets and chaffed hay (mean pellet intake 3.9 kg/d, mean hay intake 0.35 kg/d) with only a small amount of CMR (mean CMR intake of solids 0.075 kg/d). The FNZ118-fed calves continued to show a 22% reduction in CH₄ production and a 13% reduction in CO₂ production, although their H₂ production and CH₄, H₂ and CO₂ yields were no longer significantly different from the control animals. This appears to be primarily due to the 17% decrease in DMI in the FNZ118-fed calves compared to the controls.

A potential cause of lower DMI in calves could be related to low ruminal pH induced by diet, however this does not appear to have been the cause of the decrease in DMI observed in the FNZ-118 fed calves. Although some individual rumen samples (˜23%) were below 5.6 at the sampling time (2 h after feeding when rumen pH is likely to be at its lowest point), the rumen pHs were not significantly different between FNZ118-fed calves compared to the Control treatment, thereby suggesting that pH was not the cause of the lowered intake in the FNZ118-fed calves.

VFA analyses of calf rumen contents at Weeks 6 and 14 (post-weaning) did not show a significant difference between FNZ118-fed calves when compared to the control animals.

At 9 months and 1 year of age, there was a significant reduction in the total VFAs present in rumen samples from animals that had received the FNZ118 treatment, as well as the individual amounts of acetate, and butyrate, and propionate at 1 year. The amount of VFAs present in rumen samples is a balance between VFA production and VFA absorption by the rumen. Decreased amounts of VFAs in rumen samples may be due to lower VFA production (for example, because less feed is being digested) and/or increased VFA absorption by the rumen (meaning more VFAs are available for growth and development). Together with the increased body weight seen in treated animals, decreased ruminal VFA concentrations are consistent with increased absorption leading to increased feed efficiency.

There were no significant differences in body weight or average daily gain (ADG) between the FNZ118 treatment and Control calves at 6 or 14 weeks of age.

After weaning, the animals continued to show similar gains in body weight at months 4 to 6 of age. At months 7 to 10 however, the FNZ118-treated animals showed significantly more gain in body weight, and consequently their body weight was significantly higher (p<0.05) than the Control animals. From month 11 onward, the FNZ118-treated animals still showed numerically higher body weight than control animals, although this did not reach the level of significance.

ADG over the entire post-weaning period for the FNZ118-treated heifers (0.714 kg/day) was also very significantly higher (p<0.001) than for control animals (0.648 kg/day). This is despite reduced dry matter intake (measured during methane chamber measurements), suggesting that the FNZ118-treated animals were more productive and had greater feed efficiency than the control animals.

### 3.4 Conclusion

This Example shows that supplementation with L. rhamnosus FNZ118 during the first 14 weeks of life can improve the feed efficiency of animals post-weaning by supporting normal or increased growth, leading to increased body weight gain, despite reduced dry matter intake. This effect persisted to at least 1 year of age, even though supplementation had stopped at week 14 of age.

This Example also shows that L. rhamnosus FNZ118 can significantly reduce the methane production of calves, both pre-weaning (at 6 weeks of age), and post-weaning (at 14 weeks of age). Again, this effect persisted to at least 1 year of age, even though supplementation stopped at week 14.

### 4. Example 4—Impact of L. rhamnosus FNZ118 on Rumen and Faecal Microbiome

### 4.1 Materials and Methods

Rumen and faecal samples were collected at the end of the methane measurement and immediately frozen at −80° C. until DNA extraction. DNA was extracted using a bead-beating/phenol chloroform method (Rius et al., 2012) and used in PCR reactions to generate 16S ribosomal RNA gene amplicons with barcoded sequencing primers specific for bacteria, archaea and protozoa (Kittelmann and Janssen, 2011). Amplicons were purified, normalised, pooled and sequenced via an Illumina MiSeq sequencer. Sequencing results were quality controlled and filtered and the filtered sequences were analysed via QIIME using the Silva database with rumen-specific 16S rRNA gene sequences. Operational taxonomic units (OTUs) were picked at 99% similarity and tabulated.

### 4.2 Results

Samples for rumen and faecal microbiome analysis were collected at weeks 6 and 14, at the same time as methane measurements were recorded in Example 3.

### 4.2.1 Rumen Microbiome (Bacteria)

Overall, no significant differences in the rumen bacterial microbiota were observed at the phylum level between the FNZ118-treated and control animals at week 6 or week 14 (data not shown).

At the family level, there was a significant decrease (p<0.05) in the relative abundance of Ruminococcaceae at week 6 (Table 22). The difference did not persist at week 14. There was also a significant increase (p<0.05) in the relative abundance of Acidaminococcaceae at week 6, but again this difference did not persist at week 14.

TABLE 22
Relative abundance of bacterial families in the rumen microbiome.

| | Week 6 | | Week 14 | |
|---|---|---|---|---|
| Family | FNZ118 | Control | FNZ118 | Control |
| Erysipelotrichaceae | 24.1 | 19.1 | 18.8 | 19.4 |
| Prevotellaceae | 15.8 | 15.1 | 19.1 | 24.7 |
| Ruminococcaceae | 6.0 * | 10.8 | 10.7 | 10 |
| Lachnospiraceae | 13.4 | 16.6 | 22.9 | 20.3 |
| Coriobacteriaceae | 15.7 | 12.9 | 4 | 3.9 |
| Veillonellaceae | 4.3 | 4.3 | 3.9 | 2.8 |
| Christensenellaceae | 4.1 | 6.5 | 4.2 | 4.3 |
| Bacteroidales S24-7 group | 4.2 | 4 | 2.7 | 2 |
| Rikenellaceae | 4 | 4.2 | 2 | 1 |
| Mollicutes RF9 | 1.62 | 1.33 | 1.94 | 2.21 |
| Bacteroidales BS11 gut group | 1.19 | 1.23 | 0.96 | 0.7 |
| (Clostridial) Family XIII | 0.68 | 0.76 | 1.42 | 1.16 |
| Acidaminococcaceae | 1.44 | 0.63 | 1.79 | 2.5 |
| Eubacteriaceae | 0.73 | 0.54 | 0.70 * | 0.45 |
| Anaplasmataceae | 0.39 | 0.44 | 0.25 | 0.15 |
| Acidimicrobiaceae | 0.15 | 0.22 | 0.26 | 0.19 |
| Anaerolineaceae | 0.33 * | 0.18 | 0.34 | 0.45 |
| Bacteroidaceae | 0.15 | 0.16 | 0.53 | 0.9 |
| Succinivibrionaceae | 0.57 | 0.14 | 0.76 | 0.73 |
| Peptostreptococcaceae | 0.19 | 0.08 | 0.57 | 0.35 |
| Saccharibacteria | 0.02 | 0.05 | 0.02 | 0.05 |
| Family I (cyanobacteria) | 0.05 | 0.05 | 0.009 | 0.006 |
| Mycobacteriaceae | 0.015 * | 0.037 | 0.013 | 0.006 |
| Clostridiales vadin BB60 group | 0.008 | 0.035 | 0.05 | 0.033 |
| Corynebacteriaceae | 0.014 * | 0.031 | 0.006 | 0.004 |
| Porphyromonadaceae | 0.03 | 0.03 | 0.011 | 0.012 |
| Campylobacteraceae | 0.024 | 0.03 | 0.038 | 0.039 |
| Desulfovibrionaceae | 0.023 | 0.028 | 0.008 | 0.007 |
| Pasteurellaceae | 0.175 | 0.027 | 0.033 | 0.034 |
| Streptococcaceae | 0.017 | 0.02 | 0.004 | 0.009 |
| Bacteroidales RF16 group | 0.007 | 0.011 | 0.007 * | 0.003 |
| Moraxellaceae | 0.027 | 0.011 | 0.002 | 0.002 |
| Lactobacilluseae | 0.005 | 0.01 | 0.04 | 0.032 |

* p < 0.05.

At the genus level, there was a significant decrease in the abundance of Ruminococcaceae NK4A214 group and Ruminococcus 2 (bromii/bovis) at week 6. There was also a significant increase in Olsenella at week 6.

Succiniclasticum showed a significant increase in abundance at week 6 compared to the control (Table 23). Succiniclasticum is known to be involved in succinate metabolism, so this increase could indicate perturbation of the succinate metabolism pathway in the rumen.

TABLE 23
Relative abundance of bacterial genera in the rumen microbiome.

| | Week 6 | | Week 14 | |
|---|---|---|---|---|
| Genus | FNZ118 | Control | FNZ118 | Control |
| Erysipelotrichaceae UCG-002 | — | — | 3.3* | 9.2 |
| Ruminococcaceae NK4A214 group | 0.60* | 1.31 | — | — |
| Ruminococcus 2 (bromii/bovis) | 1.9* | 4.2 | — | — |
| Kandleria | 0.029 | 0.006 | — | — |
| Olsenella | 2.4* | 0.82 | — | — |
| Sharpea | 12.3 | 15.3 | — | — |
| Succiniclasticum | 1.09* | 0.45 | 1.47 | 2.13 |

*p < 0.05.

### 4.2.2 Rumen Microbiome (Archaea)

Overall, no significant differences in the rumen archaeal microbiota were observed at the family or genus level between the FNZ118-treated and control animals at week 6 or week 14 (data not shown).

At the clade level, there was a significant decrease in the abundance of an important methanogen, Methanobrevibacter gottschalkii, at week 14 in FNZ118-treated animals (Table 24).

TABLE 24
Relative abundance of archaeal clades in the rumen microbiome.

| | Week 6 | | Week 14 | |
|---|---|---|---|---|
| Clade | FNZ118 | Control | FNZ118 | Control |
| Methanobrevibacter gottschalkii | 40.6 | 46.5 | 9.1 ** | 20.8 |
| Methanobrevibacter ruminantium | 27.2 | 23.7 | 39.5 | 32.6 |
| Methanobrevibacter boviskoreani | 1.2 | 0.5 | 7.3 | 6.4 |
| Methanosphaera ISO3-F5 | 17.7 | 23.9 | 5.4 | 3.9 |

*p < 0.01.

### 4.2.3 Faecal Microbiome (Bacteria)

At the phylum level, there was a significant increase (p<0.05) in the relative abundance of Firmicutes in the faeces of animals fed FNZ118 relative to control animals at week 6 (Table 26). This effect did not persist to week 14. While L. rhamnosus FNZ118 is a member of Firmicutes, there was no corresponding significant increase in Lacticaseibacillus, suggesting that the increase in Firmicutes reflects an increase in other bacterial genera within this phylum, not just an increase in FNZ118.

TABLE 26
Relative abundance of bacterial phyla in faeces.

| | Week 6 | | Week 14 | |
|---|---|---|---|---|
| Phylum | FNZ118 | Control | FNZ118 | Control |
| Firmicutes | 66.3 * | 60.7 | 76.5 | 76.2 |
| Bacteroidetes | 28.1 | 33.2 | 17.9 | 19.2 |
| Tenericutes | 3.3 | 4.1 | 3 | 2.8 |
| Actinobacteria | 1.12 | 1.12 | 0.61 | 0.67 |
| Proteobacteria | 0.97 | 0.54 | 0.26 | 0.35 |
| Cyanobacteria | 0.03 | 0.06 | 0.09 * | 0.12 |

*p < 0.05.

At the family level, there was a significant increase (p<0.05) in the relative abundance of Ruminococcaceae, and a significant decrease in Lachnospiraceae, in the faeces of animals fed FNZ118 relative to control animals at week 14 (Table 27). There was also a significant decrease in the relative abundance of Porphyromonadaceae at week 6, although this effect did not persist to week 14.

TABLE 27
Relative abundance of bacterial families in faeces.

| | Week 6 | | Week 14 | |
|---|---|---|---|---|
| Family | FNZ118 | Control | FNZ118 | Control |
| Ruminococcaceae | 24.8 | 25.2 | 46.0 * | 42.1 |
| Lachnospiraceae | 23.9 | 20.7 | 17.4 * | 20.3 |
| Bacteroidaceae | 8.6 | 10.5 | 4.4 | 4.6 |
| Rikenellaceae | 7.2 | 9.1 | 4.6 | 5.4 |
| Bacteroidales S24-7 group | 6.8 | 5.9 | 3.9 | 4.5 |
| Porphyromonadaceae | 3.7 * | 5.6 | 0.9 | 1 |
| Mollicutes RF9 | 3.2 | 4 | 2.5 | 2.4 |
| Erysipelotrichaceae | 6.1 | 3.8 | 2 | 2.3 |
| Lactobacilluseae | 3.2 | 3.4 | 0.1 | 0.1 |
| Christensenellaceae | 3.5 | 3.1 | 6.9 | 7 |
| Peptostreptococcaceae | 2.7 | 2.1 | 0.7 | 0.8 |
| Prevotellaceae | 1.6 | 2 | 3 | 3.2 |
| Coriobacteriaceae | 1.1 | 1.1 | 0.5 | 0.5 |
| Veillonellaceae | 0.5 | 0.7 | 0.8 | 0.9 |
| Acidaminococcaceae | 0.5 | 0.5 | 0.5 | 0.6 |
| Family XIII | 0.4 | 0.4 | 1.1 | 1 |
| Clostridiaceae 1 | 0.13 | 0.19 | 0.09 | 0.21 |
| Alcaligenaceae | 0.19 | 0.19 | 0.09 * | 0.18 |
| Defluviitaleaceae | 0.11 | 0.14 | 0.26 | 0.2 |
| LWSR−14 | 0.024 | 0.106 | 0.006 | 0.002 |
| Enterobacteriaceae | 0.36 | 0.09 | 0.02 | 0.02 |
| Peptococcaceae | 0.11 | 0.09 | 0.15 | 0.17 |
| Clostridiales vadin BB60 | 0.06 | 0.09 | 0.23 | 0.26 |
| group | | | | |
| Bacteroidales UCG-001 | 0.06 | 0.09 | 0.06 | 0.07 |
| Aerococcaceae | 0.06 | 0.07 | 0.06 | 0.07 |
| Bacteroidales RF16 group | 0.07 | 0.07 | 0.48 * | 0.24 |
| Streptococcaceae | 0.103 | 0.058 | 0.003 | 0.002 |
| Mollicutes NB1-n | 0.05 | 0.045 | 0.196 * | 0.087 |
| Eubacteriaceae | 0.05 | 0.04 | 0.11 | 0.13 |
| Chloroplast | 0.02 | 0.03 | 0.05 * | 0.09 |
| Desulfovibrionaceae | 0.12 | 0.03 | 0.01 | 0.01 |
| Mollicutes EMP-G18 | 0 | 0.03 | 0.1 | 0.12 |
| Rhodospirillaceae | 0.03 | 0.03 | 0.02 | 0.02 |
| Verrucomicrobiaceae | 0.05 | 0.03 | 0.56 | 0.35 |
| Succinivibrionaceae | 0.05 | 0.03 | 0.02 | 0.03 |
| Bacteroidales BS11 gut | 0.03 | 0.02 | 0.04 | 0.02 |
| group | | | | |
| Anaerolineaceae | 0.02 | 0.02 | 0.03 | 0.03 |
| Gastranaerophilales | 0.007 * | 0.017 | 0.033 | 0.031 |
| Anaeroplasmataceae | 0.001 | 0 | 0.088 | 0.117 |
| NS9 marine group | 0.028 | 0.008 | 0.185 | 0.085 |
| Spirochaetaceae | 0.001 | 0.008 | 0.423 | 0.061 |
| Corynebacteriaceae | 0.004 | 0.008 | 0.016 | 0.06 |
| Thermoanaerobacteraceae | 0.007 | 0.011 | 0.033 | 0.057 |
| Bacteroidales Incertae | 0.001 | 0 | 0.105 | 0.051 |
| Sedis | | | | |
| Fibrobacteraceae | 0 | 0 | 0.526 | 0.037 |
| Marinilabiaceae | 0.003 | 0.003 | 0.078 | 0.037 |

* p < 0.05.

At the genus level, there was a significant increase in the abundance of Ruminococcaceae UCG-005 at week 14 in the faeces of animals fed FNZ118 relative to control animals, and a significant decrease in Ruminococcaceae UCG-014 (Table 28). There was also a significant decrease in the relative abundance of Parabacteroides at week 6.

TABLE 28
Relative abundance of bacterial genera in faeces.

| | | Week 6 | | Week 14 | |
|---|---|---|---|---|---|
| Family | Genus | FNZ118 | Control | FNZ118 | Control |
| Ruminococcaceae | Ruminococcaceae | — | — | 24.6 * | 21.4 |
| | UCG-005 | | | | |
| | Ruminococcaceae | — | — | 1.6 * | 2.2 |
| | UCG-014 | | | | |
| | Ruminiclostridium | — | — | 0.308 | 0.41 |
| | 9 | | | | |
| | Ruminiclostridium | — | — | 0.015 | 0.006 |
| | Acetivibrio | — | — | 0 | 0.001 |
| Lachnospiraceae | Epulopiscium | — | — | 0.010 * | 0.222 |
| | Lachnospiraceae | — | — | 0.069 * | 0.144 |
| Porphyromonadaceae | Parabacteroides | 2.7 * | 4.4 | — | — |
| | Porphyromonas | 0.006 * | 0.01 | — | — |

* p < 0.05.

### 4.2.4 Faecal Microbiome (Archaea)

There was no significant difference in archaeal diversity at the family, genus, or clade level in the faeces of FNZ118-treated animals compared to controls (data not shown).

### 4.3 Conclusion

This Example shows that L. rhamnosus FNZ118 can reduce the methane production of calves (as shown in Example 3), and cause a significant reduction in the ruminal abundance of an important species of methanogen, Methanobrevibacter gottschalkii, either directly through bactericidal/archaeacidal and/or bacteriostatic/archaeostatic effects, and/or indirectly through inhibiting or disrupting methanogenic pathway(s) and/or cross-feeding of intermediaries, without causing other major disruptions to the bacterial or archaeal microbiomes.

### REFERENCES

Balch, W. E., Fox, G. E., Magrum, L. J., Woese, C. R. & Wolfe, R. S., 1979. Methanogens: re-evaluation of a unique biological group. Microbiol. Rev. 43, 260-96.
Carson A F, Dawson L E R, McCoy M A, Kilpatrick D J, Gordon F J 2002. Effects of rearing regime on body size, reproductive performance and milk production during the first lactation in high genetic merit dairy herd replacements. Animal Science 74: 553-565.
De Man, J. C., Rogosa, M., & Sharpe, M. E., 1960. A medium for the cultivation of lactobacilli. J. Appl. Bacteriol. 23(1), 130-135. https://doi.org/10.1111/j.1365-2672.1960.tb00188.x
Dobos R C, Nandra K S, Riley K, Fulkerson W J, Lean I J, Kellaway RC 2001. Effects of age and liveweight at first calving on first lactation milk, protein and fat yield of Friesian heifers. Australian Journal of Experimental Agriculture 41: 13-19.
Gaspar, C., Donders, G. G., Palmeira-de-Oliveira, R., Queiroz, J. A., Tomaz, C., Martinez-de-Oliveira, J., & Palmeira-de-Oliveira, A. 2018. Bacteriocin production of the probiotic Lactobacillus acidophilus KS400. AMB Express. 8(1), 153. doi: 10.1186/s13568-018-0679-z.
Gozho G N, Plaizier J C, Krause D O, Kennedy A D, Wittenberg K M. Subacute ruminal acidosis induces ruminal lipopolysaccharide endotoxin release and triggers an inflammatory response. J Dairy Sci. 2005; 88(4):1399-403.
Krehbiel, C. R., Rust, S. R., Zhang, G., and Gilliland, S. E. (2003). Bacterial direct-fed microbials in ruminant diets: performance response and mode of action. J. Anim. Sci. 81, 120-132.
Handcock, R. C., Lopez-Villalobos, N., McNaughton, L. R., Back, P. J., Edwards, G. R., and Hickson, R. E. Positive relationships between body weight of dairy heifers and their first-lactation and accumulated three-parity lactation production. J. Dairy Sci. 102(5), 4577-4589. https://doi.org/10.3168/jds.2018-15229
Henderson G, Cox F, Ganesh S, Jonker A, Young W, Global Rumen Census Collaborators, and Janssen P H. 2015. Rumen microbial community composition varies with diet and host, but a core microbiome is found across a wide geographical range. Scientific Reports 5( )14567; doi: 10.1038/srep14567.
Hoedt, E. C., 2017. Functional and comparative studies of members of the genus Methanosphaera, and their adaptations to the gut environment. PhD Thesis, The University of Queensland, Brisbane, Australia. https://doi.org/10.1007/s002849900262
IPCC, 2014. Mitigation of Climate Change. Contribution of Working Group III to the Fifth Assessment Report of the Intergovernmental Panel on Climate Change. Cambridge University Press, Cambridge.
Jackson, R. B., Saunois, M., Bousquet, P., Canadell, J. G., Poulter, B., Stavert, A. R., Bergamaschi, P., Niwa, Y., Segers, A., Tsuruta, A., 2020. Increasing anthropogenic methane emissions arise equally from agricultural and fossil fuel sources. Environ. Res. Lett. 15, 071002. https://doi.org/10.1088/1748-9326/ab9ed2
Joblin, K. N. 1995. Methanogenic archaea. In Methods in Gut Microbial Ecology for Ruminants pp 47-53.
Kamke, J., Kittelmann, S., Soni, P., Li, Y., Tavendale, M., Ganesh, S., Janssen, P. H., Shi, W., Froula, J., Rubin, E. M., Attwood, G. T., 2016. Rumen metagenome and metatranscriptome analyses of low methane yield sheep reveals a Sharpea-enriched microbiome characterised by lactic acid formation and utilisation. Microbiome 4, 56. https://doi.org/10.1186/s40168-016-0201-2
Li, Y., Kelly, W. J., Attwood, G. T., Reid, P. and Leahy, S. C. 2019. Methanobrevibacter boviskoreani JH1T growth on alcohols allows development of a high throughput bioassay. Proceedings of the Chicago Congress on Gastrointestinal Function Chicago 2019
MacDonald K A, Penno J W, Bryant A M, Roche J R 2005. Effect of feeding level pre- and post-puberty and body weight at first calving on growth, milk production and fertility in grazing dairy cows. Journal of Dairy Science 88: 3363-3375.
Martinez-Fernandez, G., Denman, S. E., Yang, C., Cheung, J., Mitsumori, M., & McSweeney, C. S. (2016). Methane Inhibition Alters the Microbial Community, Hydrogen Flow, and Fermentation Response in the Rumen of Cattle. Frontiers in microbiology, 7, 1122. https://doi.org/10.3389/fmicb.2016.01122
McNaughton, LR and T Lopdell. 2013. Effect of heifer liveweight on calving pattern and milk production. Proceedings of the New Zealand Society of Animal Production. 73: 103-107.
Muetzel, S., Hunt, C., and Tavendale, M. H. (2014). A fully automated incubation system for the measurement of gas production and gas composition. Animal Feed Science and Technology 196, 1-11
Tyrrell, H. F., Reid, J. T., 1965. Prediction of the energy value of cow's milk. J. Dairy Sci. 48, 1215-1223. https://doi.org/10.3168/jds.S0022-0302(65)88430-2
van der Waaij E H, Galesloot P J B, Garrick D J 1997. Some relationships between weights of growing heifers and their subsequent lactation performances. New Zealand Journal of Agricultural Research 40:87-92.

### INDUSTRIAL APPLICABILITY

This invention relates to the use of probiotic bacteria, particularly L. rhamnosus strain FNZ118 and/or derivatives thereof, and in particular the use to increase feed efficiency, enhance growth and/or productivity, improve body weight or body composition of a ruminant animal, and/or increase milk production in a ruminant animal, inhibit the growth of methane-producing bacteria in the forestomach of ruminant animals, reduce the ability of the rumen microbiome to produce methane, and/or to reduce methane emissions by a ruminant animal. Methods for using L. rhamnosus strain FNZ118 and/or derivatives thereof and ruminant feed compositions comprising the same are also provided.

1. (canceled)

2. A food or feed composition comprising Lacticaseibacillus rhamnosus (Lactobacillus rhamnosus) strain FNZ118, NMIA accession number V21/015445 dated 2 Aug. 2021, or a derivative thereof.

3. The food or feed composition of claim 2, wherein the composition is a ruminant feed composition.

4. The food or feed composition of claim 2, wherein the composition is a ruminant feed composition for:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal,
h. delivering a microorganism to a ruminant animal, and/or
i. reducing the greenhouse gas emission footprint of a ruminant animal.

5. The ruminant feed composition of claim 4, wherein the feed composition:
a. is a fermented yoghurt-style composition, and wherein the fermented yoghurt-style composition is formed through a process of growing L. rhamnosus FNZ118 using a milk-based or non-milk-based carrier,
b. is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, lick block, or molasses:
c. further comprises at least one microorganism of a different species or strain, a vaccine that inhibits methanogens or methanogenesis, and/or a natural or chemically-synthesised inhibitor of methanogenesis and/or methanogen inhibitor such as bromoform; and/or
d. further comprises one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

6. (canceled)

7. (canceled)

8. (canceled)

9. The ruminant feed composition of claim 4, wherein the derivative of the L. rhamnosus FNZ 118 is a metabolite of the strain, or a culture supernatant of the strain.

10. A method for:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of a ruminant animal,
f. reducing the ability of the rumen microbiome of a ruminant animal to produce methane,
g. reducing methane emissions by a ruminant animal,
h. delivering a microorganism to a ruminant animal, and/or
i. reducing the greenhouse gas emission footprint of a ruminant animal,
said method comprising the step of administering to said animal
an effective amount of Lacticaseibacillus rhamnosus (Lactobacillus rhamnosus) strain FNZ118, NMIA accession number V21/015445 dated 2 Aug. 2021, or a derivative thereof.

11. The method of claim 10, wherein the method inhibits the growth of methylotrophic methanogens in the forestomach of the animal, optionally a methanogen from the genus Methanosphaera.

12. The method of claim 10, wherein the L. rhamnosus FNZ118 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, supplement, pharmaceutical, lick block, drench, tablet, capsule, pellet or intra-ruminal product, e.g., a bolus, or wherein the L. rhamnosus FNZ118 is encapsulated, for example in liposomes, microbubbles, microparticles or microcapsules.

13. The method of claim 12, wherein the L. rhamnosus FNZ118 or derivative thereof is administered in drinking water, milk, milk powder, milk replacement, milk fortifier, whey, whey powder, Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

14. The method of claim 10, wherein the method comprises administering to the animal L. rhamnosus FNZ118 in an amount of:
a. from 10⁴ to 10¹³ colony forming units per kilogram of dry weight carrier feed, optionally from 10⁸ to 10¹² colony forming units per kilogram of dry weight carrier feed;
b. from 10⁴ to 10¹⁰ colony forming units per kilogram of body weight of the animal per day, optionally from 10⁵ to 10⁸ colony forming units per kilogram of body weight of the animal per day; or
c. from 10⁴ to 10¹³ colony forming units per day, optionally from 10⁶ to 10¹³ colony forming units per day.

15. The method of claim 10, wherein the derivative of L. rhamnosus FNZ118 is a metabolite of the strain, or a culture supernatant of the strain.

16. The method of claim 10, the method comprising further administering at least one microorganism of a different species or strain, a vaccine that inhibits methanogens or methanogenesis, and/or a natural or chemically-synthesised inhibitor of methanogenesis and/or methanogen inhibitor such as bromoform.

17. The method of claim 10, wherein the L. rhamnosus FNZ118 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

18. The method of claim 10, wherein the method:
a. enhances the growth or productivity of the ruminant animal,
b. increases the yield of milk and/or milk components produced from the ruminant animal,
c. increases the yield of milk fat, milk protein or milk solids in milk produced from the ruminant animal, and/or
d. additionally improves the body weight and/or body composition of the ruminant animal.

19. The method of claim 10, wherein said ruminant animal is a bovine, goat, sheep, bison, yak, water buffalo, deer, camel, alpaca, llama, wildebeest, antelope, or nilgai.

20. The method of claim 10, wherein:
a. said ruminant animal is a lactating animal,
b. said ruminant animal is a pre-weaning animal, for example a calf or a lamb,
c. said ruminant animal is a post-weaning animal, or
d. the L. rhamnosus FNZ118 is administered to the ruminant animal both prior to weaning and after weaning.

21. The method of claim 10, wherein the administering is to a pre-weaning animal and wherein the inhibition of the growth of methane-producing bacteria and/or archaea in the forestomach of the ruminant animals, the reduction of methane emissions, for example methane production, by the ruminant animal, and/or the increased feed efficiency in the ruminant animal persists post-weaning.

22. The method of claim 10, wherein the inhibition of the growth of methane-producing bacteria and/or archaea in the forestomach of the ruminant animals, the reduction of methane emissions, for example methane production, by the ruminant animal, and/or the increased feed efficiency in the ruminant animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ118; or for the life of the ruminant animal.

23. (canceled)

24. A method for producing an animal product having a reduced greenhouse gas emission footprint, the method comprising:
a. providing a ruminant animal to which the method of claim 10 has been applied, and
b. producing an animal product from the animal.

25. The method of claim 24, wherein the animal product comprises dairy, meat, or wool.

26. (canceled)

27. (canceled)

28. Lacticaseibacillus rhamnosus (Lactobacillus rhamnosus) strain FNZ118, NMIA accession number V21/015445 dated 2 Aug. 2021, or a derivative thereof, for use in:
a. increasing feed efficiency in a ruminant animal,
b. enhancing the growth and/or productivity in a ruminant animal,
c. improving the body weight and/or body composition of a ruminant animal,
d. increasing the yield of milk and/or milk components produced from a ruminant animal,
e. inhibiting the growth of methane-producing bacteria and/or archaea in the forestomach of ruminant animals,
f. reducing the ability of the rumen microbiome to produce methane,
g. reducing methane emissions by a ruminant animal,
h. delivering a microorganism to a ruminant animal, and/or
i. reducing the greenhouse gas emission footprint of a ruminant animal.
